(19) United States
(12) Patent Application Publication
(10) Pub. No.: US 2025/0146074 A1
(43) Pub. Date: May 8, 2025

(54) DETERMINING WHETHER AN IBD PATIENT WILL RESPOND TO 5-ASA THERAPY

(71) Applicants: The General Hospital Corporation, Boston, MA (US); President and Fellows of Harvard College, Cambridge, MA (US); The Brigham and Womens's Hospital, Inc., Boston, MA (US)

(72) Inventors: Raaj S. Mehta, Cambridge, MA (US); Curtis Huttenhower, Cambridge, MA (US); Andrew T. Chan, Cambridge, MA (US); Jared R. Mayers, Brookline, MA (US); Emily Balskus, Cambridge, MA (US)

(21) Appl. No.: 18/939,874

(22) Filed: Nov. 7, 2024

Related U.S. Application Data
(60) Provisional application No. 63/596,703, filed on Nov. 7, 2023.

(51) Int. Cl. C12Q 1/6883; A61K 31/519; A61K 31/52; A61K 31/606; C07K 16/24; C07K 16/28

(52) U.S. Cl. CPC C12Q 1/6883; A61K 31/519; A61K 31/52; A61K 31/606; C07K 16/241; C07K 16/2842; C07K 2317/24; C12Q 2600/106; C12Q 2600/158

(57) ABSTRACT

The invention relates to a method of determining whether a patient diagnosed with inflammatory bowel disease (will respond to 5-ASA therapy which includes the steps of: obtaining a stool sample from the patient, analyzing the sample for the presence of microbial acetyltransferase genes capable of converting 5-ASA to the clinically ineffective N-acetyl 5-ASA, if microbial acetyltransferase genes are not present in the sample, the patient will respond to 5-ASA therapy and such therapy should be administered; if microbial acetyltransferase genes are present in the sample, the patient will not respond to 5-ASA therapy and a second line therapy should be administered.

### CROSS-REFERENCE TO RELATED APPLICATIONS

This application claims benefit of U.S. Provisional Application No. 63/596,703 filed Nov. 7, 2023, the contents of which are incorporated by reference.

### STATEMENT AS TO FEDERALLY FUNDED RESEARCH

This invention was made with government support under 5R35CA253185-03 awarded by the National Institutes of Health—National Cancer Institute. The government has certain rights in the invention.

### BACKGROUND OF THE INVENTION

Inflammatory bowel disease (IBD) is a chronic, debilitating gastrointestinal disorder with high rates of treatment failure.¹ At present, there is no systematic means to predict response to IBD therapy.

The anti-inflammatory drug mesalamine, also known as 5-aminosalicylic acid (5-ASA), is among the most commonly prescribed therapies available for IBD,^{2,3} and is usually formulated to be active within the colon; however, over half of IBD patients fail to respond or eventually lose response to 5-ASA over time.^{4,5} There is thus a need to identify and eliminate the causes of such treatment failure. Apropos, experimental data suggest that the gut microbiome plays a role in the pharmacokinetics of several medications.⁶⁻⁹ For example, gut microbial enzymes were recently identified and characterized in vitro to metabolize digoxin and L-dopa, medications for heart failure and Parkinson's disease, respectively.¹⁰⁻¹² In humans, association studies suggest a role of the microbiome in modulating drug efficacy of anti-cytokine biologics and cardiometabolic drugs,^{13,14} although these generally lack mechanistic explanations. As a result, few such examples of gut microbial drug metabolism have been linked both mechanistically and with clinical outcomes in humans, in the case of 5-ASA or other drugs.

Prior anaerobic stool culture experiments suggest that up to a third of 5-ASA can be metabolized by gut bacteria into N-acetyl 5-ASA,^{15,16} a compound that lacks anti-inflammatory activity in placebo-controlled trials.^{17,18} This is at least in part due to the metabolite's diminished (<5%) bioavailability to colonic epithelial cells.¹⁹ Experimental work has identified several bacteria capable of 5-ASA metabolism,^{20,21} but the majority of these are typically absent or of low abundance in patients with IBD. In any case, the specific microbial enzyme(s) responsible for the inactivation of 5-ASA in the gut microbiome have remained elusive for the last forty years. Additionally, prior to this work, the clinical implications of the microbial enzyme(s) which inactivate 5-ASA were unclear.

### SUMMARY OF THE INVENTION

In one aspect, the invention features a method of determining whether a patient diagnosed with inflammatory bowel disease (IBD) will respond to 5-ASA therapy, the method including:
(a) obtaining a stool sample from the patient,
(b) analyzing the sample for the presence of microbial acetyltransferase genes capable of converting 5-ASA to the clinically ineffective N-acetyl 5-ASA,
(c) if microbial acetyltransferase genes are not present in the sample, the patient will respond to 5-ASA therapy and such therapy should be administered; if microbial acetyltransferase genes are present in the sample, the patient will not respond to 5-ASA therapy and a second line therapy should be administered.

In some embodiments, the microbial acetyltransferase genes are three or more of the following genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii.

In some embodiments, IBD is ulcerative colitis. In other embodiments, IBD is Crohn's Disease. In yet other embodiments, the second line therapy is selected from a biologic for treating IBD. In still other embodiments, the second line therapy is selected from an immunomodulator for treating IBD. In another embodiment, analyzing the stool sample includes employing quantitative polymerase chain reaction (qPCR).

In another aspect, the invention involves treating a patient having IBD which includes administering to the patient a biologic in an amount effective to treat IBD when a stool sample from the patient includes microbial acetyltransferase genes capable of converting 5-ASA to N-acetyl 5-ASA. In one embodiment, IBD is ulcerative colitis. In another embodiments, IBD is Crohn's Disease. In still another embodiment, the biologic for treating IBD is adalimumab, vedolizumab, orinfliximab.

In another aspect, the invention involves treating a patient having IBD which includes administering to the patient an immunomodulator in an amount effective to treat IBD when a stool sample from the patient includes microbial acetyltransferase genes capable of converting 5-ASA to N-acetyl 5-ASA. In one embodiment, IBD is ulcerative colitis. In another embodiments, IBD is Crohn's Disease. In still another embodiment, the immunomodulator for treating IBD is 6-mercaptopurine or tofacitinib.

In another aspect, the invention features a method for treating a patient having IBD includes administering to the patient 5-aminosalicylic acid (5-ASA) in an amount effective to treat IBD when microbial acetyltransferase genes are not present in a stool sample from the patient. In one embodiment, IBD is ulcerative colitis. In another embodiments, IBD is Crohn's Disease. In one embodiment, wherein the microbial acetyltransferase genes are three or more of the following genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii.
   In some embodiments, determining presence or absence of microbial acetyltransferase genes in the stool sample involves a qPCR methodology.

In any of the aforementioned aspects and embodiments, the patient is a human patient.

Other features and advantages of the invention will be apparent from the following detailed description and figures, and from the claims.

### BRIEF DESCRIPTION OF THE DRAWINGS

The patent or application file contains at least one drawing executed in color. Copies of this patent or patent application with color drawings will be provided by the Office upon request and payment of the necessary fee.

FIG. 1 shows the identification of microbial 5-ASA inactivating enzymes from IBD microbiome population multi-omics. As part of the HMP2 IBDMDB (timeline, upper left),²² 132 participants with and without IBD were followed for 1 year, each completing multiple dietary and medication questionnaires, and each providing stool every two weeks and blood samples approximately quarterly. After excluding participants without IBD or without metabolomics data, we identified 45 verified users of 5-ASA in the cohort and 34 non-users. Among 5-ASA users, we found 13 individuals who started or resumed using the drug during the cohort follow-up. Stool from >1,000 samples was then profiled through metagenomics, metatranscriptomics, and/or metabolomics; blood was analyzed by exome sequencing which was ultimately leveraged to determine human NAT2 acetylation phenotypes (“fast” vs. “slow”) for our clinical exploration. In the analysis phase, we first studied the impact of 5-ASA on the fecal metabolome and then identified gut bacterial enzymes involved in inactivating 5-ASA to N-acetyl 5-ASA. Finally, we related these enzymes to risk of disease relapse.

FIG. 2 shows that 5-ASA directly impacts the fecal metabolome and undergoes biotransformation by the microbiome. (A) The IBD fecal metabolome segregates by 5-ASA status (PERMANOVA R2=6.8%, p<0.001) more than by UC (ulcerative colitis) or CD (Crohn's Disease) diagnosis (R²=2.2%), suggesting a substantial role of medication in modulating the fecal biochemical environment of IBD patients (95% bivariate normal confidence ellipses shown, Methods). (B) Initiation of 5-ASA among a subset of participants (n=13) reveals 2,306 (total n=81,868, 2.8%) altered metabolomic features when comparing profiles pre- and post-5-ASA administration (paired Wilcoxon, FDR q<0.25), collected an average of 13.0 (±8.7) weeks apart. Only 17 were assigned Human Metabolome Database (HMDB) identifiers, including the known 5-ASA metabolite, N-acetyl 5-ASA, as well as potential off-target effects—including shifts in vitamin B3 metabolism and bacterial products implicated in oxidative stress (23, 24). **, q<0.05; *, q<0.25; NS, not significant. (C). Examining the remaining 2,293 unannotated metabolomic features, we identified two promising candidates in the IBDMDB and independently profiled PRISM datasets using mass differences and retention time matching as likely N-propionyl 5-ASA and N-butyryl 5-ASA that also discriminated 5-ASA users from non-users (c-statistic >0.95) that were not initially annotated by the HMDB (Methods).

FIG. 3 shows that microbial genes are metatranscriptomically implicated in generation of N-acetyl 5-ASA cluster into thiolase and acyl CoA N-acyltransferase superfamilies. (A) MTX abundance distributions for six putative 5-ASA-acetylating gene families in 5-ASA users vs non-users. Multivariate linear mixed effects models adjusted for DNA copy number (27) identified two significantly overexpressed gene clusters with putative acetyltransferase function in 5-ASA users vs. non-users: 1) a GNAT family N-acetyltransferase (UniRef90 ID: C7H1G6) and 2) an acetyl-CoA acetyltransferase (UniRef90 ID: R6TIX3) (FDR q0.24 and 0.14, respectively). Searching for any additional sequences with at least 80% full-length sequence similarity yielded three additional hits, all from the first acetyl-CoA acetyltransferase, each nominally enriched in 5-ASA users compared to non-users. (B) Specificity and sensitivity for microbial transcripts with respect to presence/absence of fecal N-acetyl 5-ASA across samples from 5-ASA users identifies seven additional putative 5-ASA acetylating gene families. In our second criteria, we estimated sensitivity and specificity for how each metatranscriptomic gene cluster (presence/absence) detected dichotomized N-acetyl 5-ASA (high/low). Using a 50% cutoff (hashed line) for each test characteristic revealed an additional 7 putative acetyltransferase gene clusters. Shown outside of these bounds are results from the first criteria, highlighting high degrees of specificity, but lower sensitivity. (C) MTX relative abundances for three of these MBX-based candidates identified in the second criteria also correlated with fecal N-acetyl 5-ASA levels (R2 and p values inset). (D) Pooled metatranscriptomic families from parts A and B proved to cluster into two protein superfamilies—thiolase and acyl-CoA N-acyltransferases—which are carried primarily by Bacteroides and Firmicutes phylum members, respectively. (E). Multiple sequence alignment of these twelve candidate enzymes shows highly conserved sequences among the thiolase enzymes (green) but greater diversity among acyltransferases (light blue). (F) In genomes from isolate strains, the only acyl-CoA N-acyltransferase gene carried by a Firmicutes member was in a subset of F. prausnitzii strains. When hierarchically clustered according to a prior schema³³ (repurposed with permission), these appeared to originate from only one of the clade's major phylogroups, suggestive of acquisition via a horizontal transfer event.

FIG. 4 shows that heterologous expression and purification of a gut microbial acetyltransferase confirms 5-ASA acetylation activity in vitro. (A) A predicted thiolase (R6CZ24) from an uncultured Firmicutes (FcTHL) and a predicted acyl CoA N-acyltransferase (C7H1G6) from F. prausnitzii (FpGNAT) convert 5-ASA to N-acetyl-5-ASA in the presence of acetyl-CoA. Reactions were carried out for 6 hr at 37° C. with 1 mM of each substrate and 50 μM enzyme. (B) An in vitro pooled acyl-CoA competition assay with 1 mM of each acyl-CoA species, confirms varying length acyl groups can be donated to 5-ASA, as detected in our analysis in patients with IBD (FIG. 2c) (****=p<0.0001, *=p<0.05). (C) Tetrameric crystal structure of FcTHL, shown as a ribbon diagram with one dimer in purple and orange and the other dimer in green and blue. Two tightly interacted dimers form a tetramer through an L-domain. (D) Alignment of an acetylated monomer from the FcTHL with an acetylated biosynthetic thiolase monomer (1 DM3) in complex with acetyl CoA from Zoogloea ramigera (ZrTHL) highlights good agreement and reveals an overlapping Cys-His-Cys catalytic triad poised to acetylate a substrate. (E) Using a similar method of protein structure superimposition, the crystallized Salmonella enterica typhimurium NAT (PDB ID: 1 E2T, SeNAT, known to acetylate 5-ASA) and the Firmicutes thiolase have very different overall structures, yet both enzymes' active sites contain cysteine and histidine residues in similar positions and perform similar reactions (FIGS. 16 a-b).

FIG. 5 shows that gut microbial 5-ASA-inactivating acetyltransferases are associated with greater risk of treatment failure in 5-ASA users. (A) Bacterial acetyltransferase genes are associated with an increased risk of steroid use. Red squares with labels represent odds ratios, with 95% confidence intervals represented by gray bars. (B) Metagenomic carriage of 3-4 acetyltransferases (identified in panel A) compared to those with 0-2 have an increased risk of steroid use in the IBDMDB. In an independent, prospective validation cohort, SPARC IBD, baseline carriage of the same 3-4 acetyltransferases confers a similarly increased risk of future steroid use. Pooled analysis demonstrates consistent effect size across the two cohorts. Studies are inversely weighted according to their variance. (C) Gene prevalence varies across participants with IBD but does not vary by 5-ASA status (GLMM, p≥0.07), arguing for effect modification of the effects of 5-ASA on prevention of disease relapse.

FIG. 6 shows that 5-ASA levels according to self-reported 5-ASA use in the IBDMDB. We determined 5-ASA use according to detection of drug levels in stool using LC-MS, defined as detection of fecal 5-ASA levels >10e7. Concordance, determined according to statistical accuracy (Methods), between self-reported use of 5-ASA and detection of fecal 5-ASA was 80.3% (FIGS. 14 and 15). Users (light blue) had 5-ASA levels which were ˜10,000× greater than non-users (navy blue). Each individual is represented by multiple points on this graph given that participants provided multiple samples across the year-long cohort.

FIG. 7 shows that 5-ASA associated metabolites predictably rise and fall with initiation and discontinuation of the medication. Time series for a 10-year-old boy with newly diagnosed Crohn's colitis in the IBDMDB who had a stuttering disease course throughout the year-long study. Initially started on Pentasa—an ethylcellulose-coated, controlled-release of oral 5-ASA—he experienced a flare and was switched to prednisone. Ultimately, he resumed 5-ASA—Lialda, a multimatrix formulation of 5-ASA—but flared again and was escalated to infliximab and methotrexate. With each of these starts and stops, 5-ASA-associated metabolomic features predictably sunk and surged, even with administration of two different formulations. Abbreviations: HBI, Harvey-Bradshaw Index (metric of disease activity).

FIG. 8 shows representative indirect effects of 5-ASA on the fecal metabolome include shifts in Vitamin B3 and its metabolism towards glycine conjugation and formation of nicotinuric acid. We considered that 5-ASA may lead to highly specific markers of medication use mediated through indirect microbial pathways, such as promotion of growth of certain bacteria which, in turn, generate compounds. Here, we find that the abundance of nicotinic acid (NA) dropped as nicotinuric acid (NUA) dramatically increased; there was no consistent change in the third niacin metabolite detected on our platform, M-methyl-nicotinamide. While the precise role of 5-ASA in determining the fate of NA is unknown, for >70 years, anaerobic bacteria, including Clostridia species have been known to metabolize NA (24). Furthermore, medications, such as aspirin, are known to affect the balance of NA and NUA in the blood.⁷⁹ Intriguingly, fecal NA levels have previously been detected at lower levels in IBD patients compared to healthy controls,⁸⁰ but confounding by 5-ASA use was not explored. Given the role of anaerobic gut bacteria in metabolism of NA, it is conceivable that gut bacteria promoted by 5-ASA- or 5-ASA itself—shunts NA towards the glycine conjugation pathway without profound impact on amidation. This phenomenon was not seen in initiators of steroids or biological drugs in the cohort; NUA levels were undetectable in non-5-ASA users.

FIG. 9 shows that 5-ASA use is associated with large-scale differences across individual gut microbial species abundance in patients with IBD. Significant associations (n=24, FDR q<0.25) of 5-ASA use and microbial species overlaid onto their taxonomy. The blue-to-orange gradient in smaller circles represents the magnitude and direction of the associations between 5-ASA and species' abundances. Here we only show a subset (dark orange, dark blue) of the strongest associations discovered. The shading in the figure differentiates major phyla. All models included each participant's identifier as random effects and simultaneously adjusted for age, disease type, use of antibiotic, and dysbiosis status.

FIG. 10 shows the impact of 5-ASA on the fecal metabolome is influenced by the gut microbiome. A) Drug levels, followed by gut microbiome taxonomic profiles, independently explain variation in most 5-ASA-derived metabolite levels. These were analyzed systematically by linear models constructed for each metabolite with independent variables being: fecal 5-ASA levels; microbiome data; host data, including diet, disease type, age, other medications, and sex; and other/unexplained. We quantified variance explained (EV) by each model and partitioned EV by each term for each 5-ASA-shifted metabolite (shown along the x-axis, as a stacked proportional bar plot). As an example, N-acetyl 5-ASA had a moderate correlation with 5-ASA levels in stool (Spearman rho 0.50, p=4e-9), with 35% of variance explained by drug levels, 15% explained by the microbiome, and 7% explained by other host features. B) Associations between differentially abundant (DA) metabolites and DA species using HAIIA (Methods). Block associations are numbered in descending order of significance (max FDR<0.25), with each numbered block corresponding to a group of co-occurring metabolites with a species. A white dot indicates marginal significance of a particular pair of features (p<0.05). C) Abundance of R. inulinivorans was inversely correlated with the metabolite peak 242.0458 (represented by Block 17 in (B)), which was 37,661-fold greater on average among users than non-users (FDR 0.001). D) F. prausnitzii was positively associated with the peak 373.1254 (represented by Block 20 in (B)), which was 73-fold greater among users than non-users (FDR 0.001).

FIG. 11 shows that fecal 5-ASA levels are concordant with self-reported 5-ASA use in PRISM. As in HMP2, there was high accuracy between self-reported use of 5-ASA with detection of fecal 5-ASA. We used the PRISM cohort, an independent cohort of patients with IBD with untargeted fecal metabolomics, to validate our annotation of 5-ASA derivatives beyond N-acetyl 5-ASA.

FIG. 12 shows that microbial species previously characterized to acetylate 5-ASA have low or absent representation in the gut microbiomes of patients with IBD. A) Phylogenetic analysis shows the taxonomic contributions to the MTX (purple, n=301 species) and MGX (gold, n=322 species) data in the IBDMDB. Also shown are bacteria (green, n=20 species) previously found to acetylate 5-ASA in vitro,¹⁸ all of which belong to the Proteobacteria phylum. Notably, only six of these had detectable MGX and MTX levels in the HMP2, which are labeled and listed. B) The abundance of Proteobacteria is low across the vast majority of participants, relative to Firmicutes and Bacteroidetes species. C) The prevalence of the six species with potential for 5-ASA acetylation activity is low, except E. coli. Most importantly, none of these six bacterial species have detectable arylamine N-acetyltransferase enzymes at the metatranscriptomic level in the IBDMDB.

FIG. 13 shows that gut microbiome genes were prioritized for experimental characterization via a three-pronged multi-omics approach. We took three-pronged approach to identifying 5-ASA metabolizing enzymes; each part was independent of the other. In Part 1, we used two arylamine N-acetyltransferase (NAT) sequences from Salmonella enterica serovar typhimurium LT2 (nhoA) (UniProt accession, Q00267)²⁰ and Pseudomonas aeruginosa²¹ (UniProt accession, Q9HUY3), previously shown to metabolize 5-ASA, as well as 105 NAT or N-hydroxyarylamine 0-acetyltransferase microbial protein sequences predicted to metabolize 5-ASA as the query for a BLASTP search (Diamond v0.9.24.125) of the Human Microbiome Project (HMP) reference isolate genomes with an e-value of 10 down to the 25% identity. Given that there were no hits at the metatranscriptomic level, in Part 2, we then used differential abundance testing to identify significantly overexpressed acetyltransferases, fit with a per-feature linear mixed-effects model, which adjusted for DNA copy number, which allows for biological and technical zero values while also controlling for underlying DNA levels (q<0.25). These two hits were expanded at 80% homology to find similar functional proteins. In Part 3 of our approach to identifying 5-ASA metabolizing enzymes, we compared metatranscriptomic abundance of individual gene families (present/absent) with the metabolomics readout of dichotomized N-acetyl 5-ASA (high/low) in each stool sample, and then derived measures of sensitivity (true positives/(true positives+false negatives)) and specificity (true negatives/(true negatives+false positives)) for each gene cluster to estimate how well a given cluster correlates with the drug metabolite. All of these were pooled to arrive at 12 candidates.

FIG. 14 shows that heterologous expression of thiolase and acyl-CoA N-acyltransferase enzymes in E. coli BL21. (A) Coomassie-stain gel shows purity of indicated enzymes after overexpression and cobalt purification. Abbreviations: FcTHL=a predicted thiolase (R6CZ24) from an uncultured Firmicutes; FpGNAT=a predicted acyl CoA N-acyltransferase (C7H1G6) from F. prausnitzii; StNAT=a known arylamine N-acetyltransferase from Salmonella enterica serovar typhimurium LT2. (B) Confirmation of in vitro acetylation of 5-ASA by known S. typhimurium enzyme using 1 mM of each substrate and 5 uM enzyme incubated for 1 hr at RT.

FIG. 15 shows extended biochemical data for the 5-ASA metabolizing thiolase enzymes. (A) In vitro competition assay with 1 mM of 5-ASA, 4-ASA, procainamide, hydralazine and isoniazide demonstrates relative specificity of the Firmicutes CAG:176 thiolase (FcTHL) for 5-ASA. (B) Shown is a representative example of one of five independent kinetics experiments (each concentration was performed in biological triplicate in each repeat). (C) Live culture of Oscillibacter sp., strain KLE 1745 encoding another predicted thiolase gene (UniRef90 R6TIX3) was capable of acetylation of 5-ASA to N-acetyl 5-ASA.”

FIG. 16 shows a comparison of canonical acetyltransferase reaction mechanisms for thiolase and arylamine NAT enzymes, now both shown to acetylate 5-ASA. A) The thiolase two-step “ping pong” mechanism (Modis) begins after a cysteine is activated by a nearby histidine residue and then performs a nucleophilic attack on an acetyl CoA molecule to form a covalent acetyl-enzyme intermediate (shown in FIG. 4d). In the second step, the substrate (classically a second acetyl CoA molecule) nucleophilically attacks the acetyl-enzyme intermediate to yield the final acetyoacetyl-CoA and enzyme. The second nucleophilic attack is activated by a second cysteine residue in the active site, which deprotonates the substrate. B) In a similar two-step “ping pong” mechanism in the arylamine NAT, a cysteine is also activated by a nearby histidine residue and then performs a nucleophilic attack on an acetyl CoA molecule to form a covalent acetyl-enzyme intermediate. In contrast, rather than abstracting a proton from another cysteine residue in the active site, the departing CoA molecule deprotonates the histidine residue, which allows an arylamine substrate to perform nucleophilic attack on the acetyl-enzyme intermediate to yield the final acetylated substrate and enzyme. C) We speculate that in some cases, an acetyl-thiolase enzyme intermediate is formed which may allow nucleophilic attack by an arylamine, such as 5-ASA.

FIG. 17 shows a comparison of the crystalized Firmicutes CAG:176 thiolase (FcTHL) with an acetylated thiolase crystal structure in complex with acetyl CoA from the well-characterized gram-negative Zoogloea ramigera (ZrTHL, PDB ID: 1dm3) highlights agreement between monomers ([RMSD]1.652)

FIG. 18 shows an overview of the Study of a Prospective Adult Research Cohort with IBD (SPARC IBD) study. A) Timeline shows the stool sampling scheme in relation to clinical assessments throughout the cohort. At study entry, all participants provided a single stool sample. The median time to event (use of steroids) was 229 days. Among a minority of participants (n=33) who voluntarily provided additional stool samples throughout the cohort (denoted by empty blue circles, Methods), median interval sampling time was 133 days. B). Flow diagram illustrating inclusion and exclusion criteria for this analysis. C) In a sensitivity analysis, we limited our analysis to a single (baseline) stool sample per participant. With diminished power, the SPARC IBD-specific estimate was no longer significant (OR 2.22, 95% CI 0.74-6.69). Accordingly, age and CD were not significantly associated with risk of steroids despite being established risk factors for 5-ASA treatment failure.³⁹ Nevertheless, the pooled meta-analysis was not meaningfully different from the primary analysis (OR 2.78, 95% CI 1.19-6.50).

FIG. 19 shows a validity analysis of 5-ASA annotation by metabolomics methods. Two separate standards for 5-ASA (SIGMA catalog PHR1060 and 18858) confirmed the identity of the 5-ASA peak in the IBDMDB (m/z: 154.0502, RT: 3.83 min) through retention time (panel A) and spectral matching (panel B).

FIG. 20 shows a validity analysis of N-acetyl 5-ASA annotation by metabolomics methods. A) A standard for N-acetyl 5-ASA (Cayman Catalogue 27618) produced two peaks which matched two peaks in the IBDMDB (QI3818 and QI3816). The later eluting peak (QI3816), was more abundant in the IBDMBD stool, correlated better with 5-ASA (Pearson's correlation r=0.89 vs r=0.60), and B) had excellent MS/MS spectral matching. Further still, levels of QI3816 perfectly discriminate 5-ASA users from non-users (c-statistic 0.99).

### DETAILED DESCRIPTION OF THE INVENTION

As is described below, we have identified and biochemically validated gut microbial enzymes capable of generating the clinically ineffective 5-ASA derivative N-acetyl 5-ASA. In turn, we show that the presence of these microbial acetyltransferase genes in gut metagenomes is associated with an increased risk for 5-ASA treatment failure. These findings were driven and validated by metagenomics (MGX), metatranscriptomics (MTX) and metabolomics (MBX) from more than 1,000 stool samples collected over a 1-year period from patients with IBD and controls²² as well as subsequent phylogenetics, heterologous expression and chemical characterization of the target proteins.

Dense Time-Series Multi-Omic Profiles from an IBD Patient Cohort Reliably Identify 5-ASA Use and Chemical Derivatives

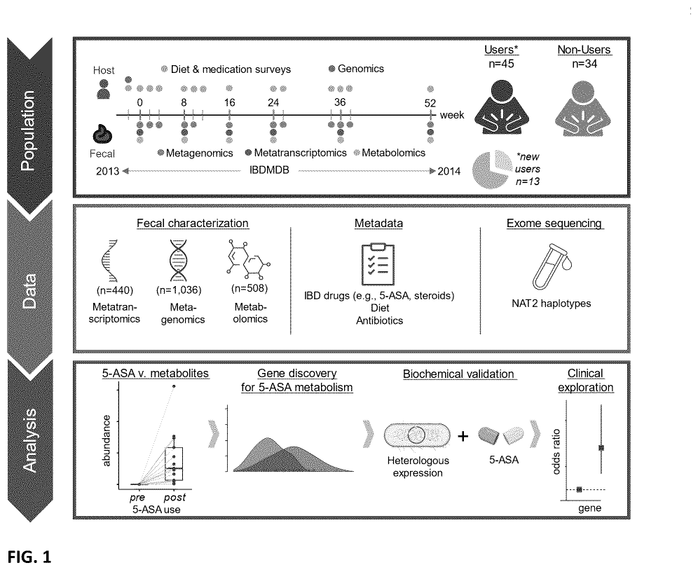

To study the role of the gut microbiome in metabolizing 5-ASA and modulating its efficacy, we leveraged data from the Integrative Human Microbiome Project (iHMP or HMP2) Multi-omics Database (IBDMDB, ibdmdb.org), a multi-center cohort of 132 individuals with and without IBD, who each provided repeated medication, dietary, and symptom assessments along with serial stool and blood samples over one year (FIG. 1 and Methods).²² Stool samples from 79 participants with Crohn's Disease (CD) or ulcerative colitis (UC) were newly profiled by the bioBakery 3 suite,²³ yielding 1,036 metagenomes (MGX), 440 metatranscriptomes (MTX), and 508 untargeted metabolomes (MBX), with 283 MBX-MGX pairs and 213 MGX-MTX pairs.

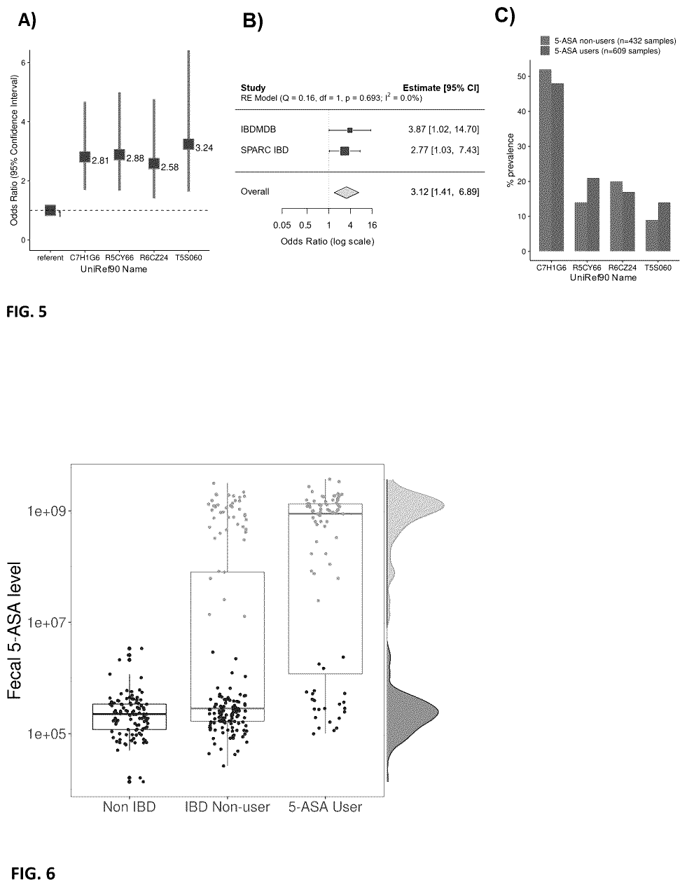

Concordance between self-reported use of 5-ASA (Methods) and detection of fecal 5-ASA was 80.3% (FIG. 6), a rate that was consistent with other population-based cohort studies.²⁴ To avoid misclassification in self-reported data, we assigned 5-ASA user status according to detection of drug levels in stool drug (Methods). Users had 5-ASA levels which were ˜10,000× greater than non-users.

Consistent with clinical practice,²⁵ participants on 5-ASA were more likely to have UC than CD, and less than 10% were users of bonded 5-ASA (sulfasalazine, olsalazine, or balsalazide) (Table 1). Of note, 13 individuals were found to start or resume 5-ASA therapy during their sampling time courses, termed “new users”—providing an opportunity to examine the direct impact of the drug on the microbiome. Samples pre- and post-5-ASA administration were collected an average of 13.0 (±8.7) weeks apart.

### 5-ASA Directly Shapes the Fecal Metabolome and Undergoes Biotransformation by the Gut Microbiome

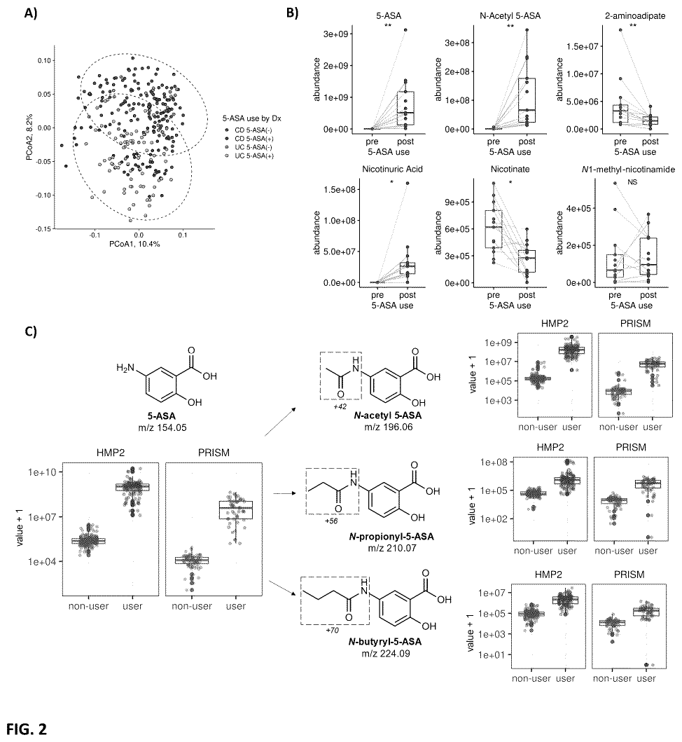

Fecal metabolomic profiles segregated significantly according to 5-ASA user status (FIG. 2a, PERMANOVA R²=6.8%, p<0.001). Interestingly, this separation was greater than that due to baseline IBD diagnosis (e.g., CD vs. UC, R2=2.2%, p<0.001). Here, 5-ASA status may serve as a surrogate for metabolic differences driven by disease severity: “ever-users” vs. “never-users” of the drug were less likely to be hospitalized during the study and trended towards lower rates of bowel surgery (Table 1).”

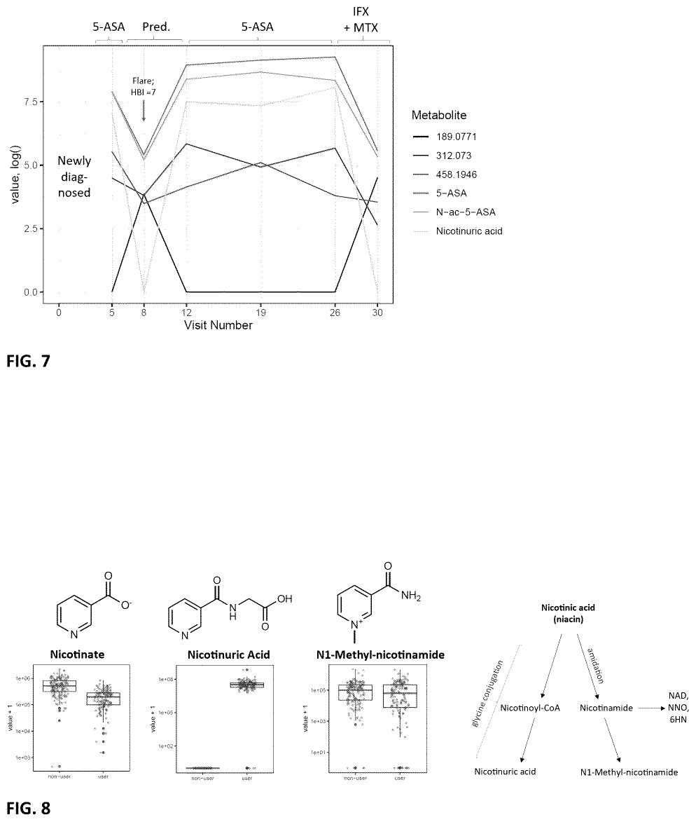

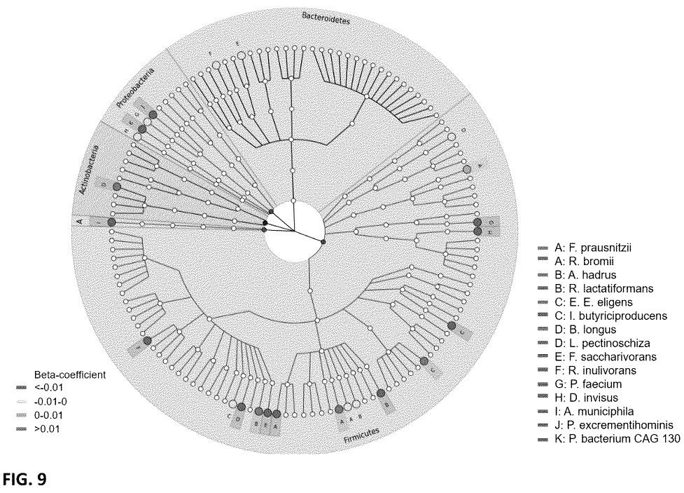

To identify specific fecal metabolites directly modulated by 5-ASA use, we focused our attention on the natural experiment of 13 new users of 5-ASA, defined as those patients for whom there were pre- and post-treatment stool samples. 2,306 metabolomic features (2.8%, n=81,868) were significantly differentially abundant when comparing stool pre- and post-5-ASA (paired Wilcoxon, FDR q<0 (FIG. 7), of which only 17 were confidently assigned to Human Metabolome Database (HMDB) identifiers. As expected, these included 5-ASA and N-acetyl 5-ASA, but also potential microbial mediators of the anti-inflammatory effects of the drug. For example, the abundance of 2-aminoadipate, a bacterial metabolite in the lysine synthesis pathway²⁶ linked to greater oxidative stress,²⁷ was reduced following 5-ASA treatment, which may represent another way 5-ASA exerts its anti-inflammatory effects (FIG. 2b). In addition, we found that 5-ASA induced large shifts in niacin metabolism, known to occur in anaerobic gut bacteria,²⁸ and previously seen to modulate inflammation in ulcerative colitis²⁹ (FIG. 2b; FIG. 8). These effects were highly specific to 5-ASA use and could not be attributed simply to suppression of inflammation: in a subset of 9 participants in the IBDMDB who started biologic drugs, there was no change in nicotinic acid levels, and nicotinuric acid levels were undetectable in non-users of 5-ASA (data not shown).

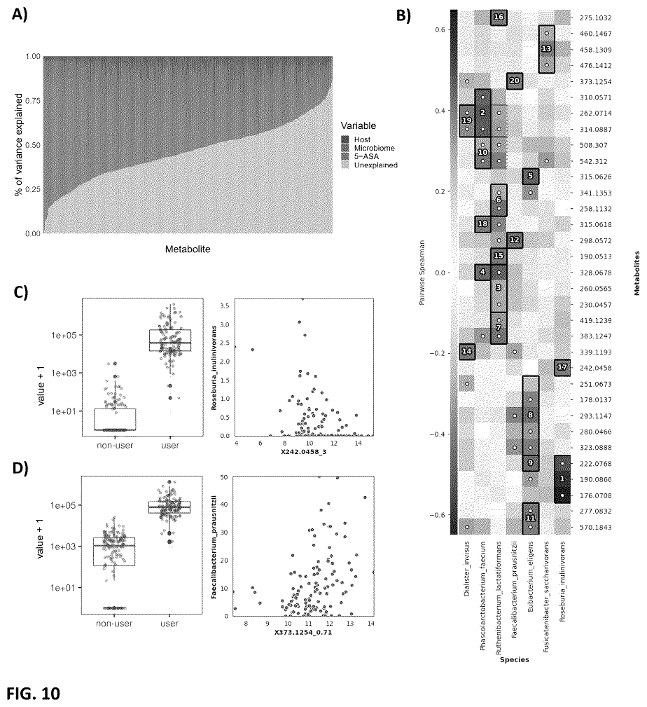

We next sought to estimate the relative contributions of microbial, host, and other factors to these differentially abundant metabolites, given that many of these factors will co-vary during 5-ASA use. In models containing fecal 5-ASA levels; microbiome taxonomic data; host variable data, including diet, disease type, age, other medications, and sex; and an other/unexplained term, we quantified variance explained (EV) in each 5-ASA-shifted metabolite. As expected, by definition, 5-ASA drug levels had the largest predictive power (mean ˜29% EV) in determining 5-ASA-modulated metabolite levels, followed by microbiome features (median ˜19% EV) and other host factors (FIG. 10, Methods). As an example, N-acetyl 5-ASA, moderately correlated with 5-ASA levels in stool (p 0.50, p=4e-9), with 35% of EV by fecal 5-ASA levels, 15% by the microbiome, and 7% by other host features.

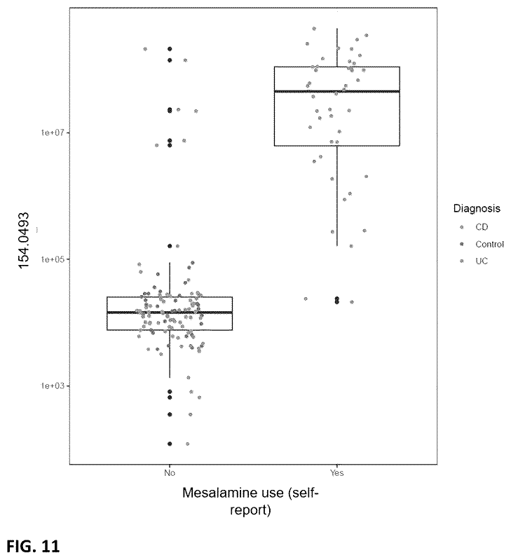

We then wanted to determine if any of the remaining 2,293 unannotated metabolic features represented unrecognized biotransformations of 5-ASA. Specifically, we calculated differences between the masses of significantly altered metabolites and 5-ASA, and then mapped these against mass shifts of known microbial biotransformations.⁸ As a positive control, we started with N-acetyl 5-ASA. As expected, the mass shift corresponded to acetylation (+42), and this peak distinguished 5-ASA users from non-users in the IBDMDB essentially as well as the parent compound (c-statistic 0.99) (FIG. 2c). This finding was further replicated in another independent cohort of IBD patients (PRISM)⁸ (FIG. 11), as was the identification of two previously unannotated likely 5-ASA derivatives, N-propionyl 5-ASA and N-butyryl 5-ASA (FIG. 2c, Table 2). Thus, in combination with large-scale impacts of 5-ASA on the gut metabolome, we consistently identified several 5-ASA acyl-derivatives in stool metabolomes and found evidence that their variance can be explained, at least in part, by the gut microbiome.

### Identification of Enzymes Putatively Responsible for 5-ASA Biotransformation in Human Gut Microbiome Assemblies

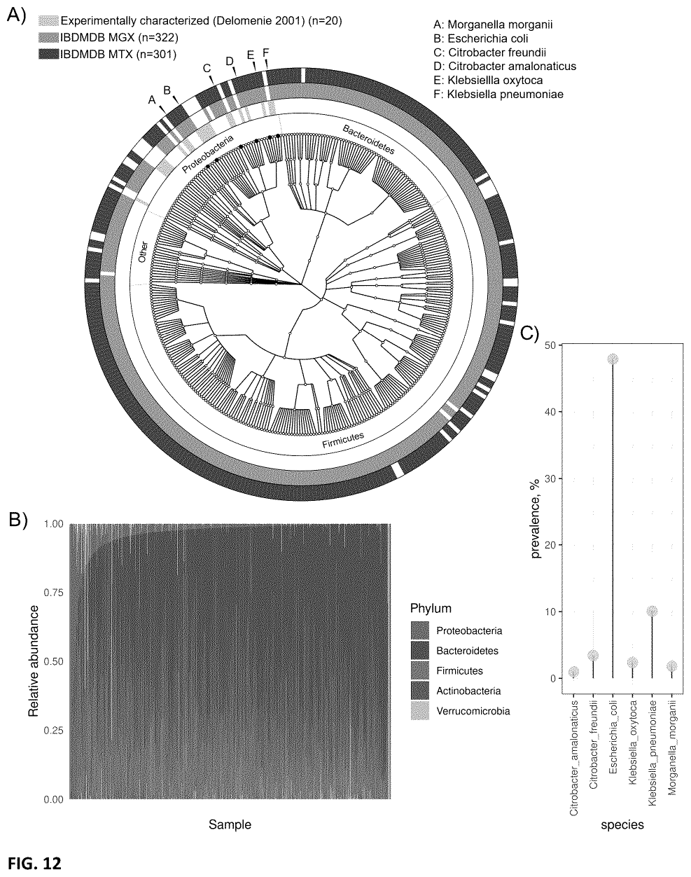

Next, we sought to identify which gut microbial enzymes were involved in generating the clinically ineffective metabolite N-acetyl 5-ASA, as well as the other putative biotransformations of 5-ASA observed above. Notably, typical homology-based approaches^{6,9,11} were not able to provide any candidates, even using very generous thresholds over more than a thousand metagenomes. Specifically, using two arylamine N-acetyltransferase (NAT) sequences from Salmonella enterica serovar typhimurium LT2 (nhoA) and Pseudomonas aeruginosa, previously shown to metabolize 5-ASA,^{20,21} as well as 105 NAT or N-hydroxyarylamine O-acetyltransferase microbial protein sequences predicted to metabolize 5-ASA, we queried the complete set of gut microbial proteins in the IBDMDB HUMAnN 3-formatted UniRef90 database at a minimum of 25% full-length homology. This matched 5,685 unique gene clusters. Only 16 of these gene families were also detected in the IBDMDB gut metagenomic profiles, 14 of which mapped to E. coli and 2 of which were NATs. Notably, none were present in our metatranscriptomic data. As expected, there was no detectable Salmonella or Pseudomonas aeruginosa found in the gut of IBD patients. Furthermore, bacterial species previously found to acetylate 5-ASA were absent or of low abundance and/or prevalence in the gut microbiome (FIG. 12) and did not express NAT genes.

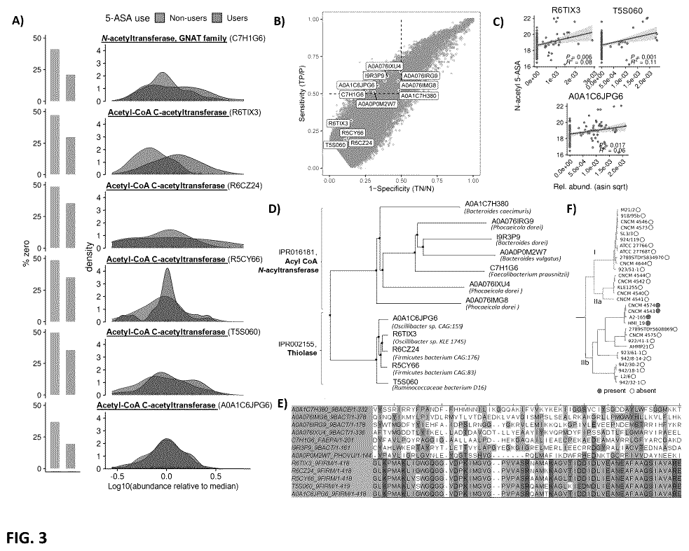

This prompted the development of our novel multi-omic criteria, combining 1) metatranscriptomics- and 2) metabolomics-based strategies. To first derive candidates from MTX profiles, we performed multivariate testing for differentially expressed microbial transcript families between 5-ASA users and non-users, accounting for DNA copy number³¹ (Methods). We identified two significantly overexpressed UniRef90 gene clusters with putative acetyltransferase function: 1) a GNAT family N-acetyltransferase (UniRef90 ID: C7H1G6) and 2) an acetyl-CoA acetyltransferase (UniRef90 ID: R6TIX3) (linear mixed effects models, β 0.0003, FDR q0.24 and β 0.0003, FDR q 0.14, respectively) (FIG. 3a). We then re-extended our search using sequence similarity to these two novel hits, identifying proteins sharing >80% amino acid identity to retain conserved function.³² We identified four additional candidates with putative acetyltransferase domains for the acetyl-CoA acetyltransferase hit (UniRef90 IDs: R6CZ24, R5CY66, T5S060, A0A1C6JPG6) that were nominally enriched at a metatranscriptomic level between 5-ASA users vs. non-users (p=0.05, p=0.02, p=0.04, p=0.006 respectively), but not significant in our first search. There were no expanded candidates for the GNAT family N-acetyltransferase.

In our second, MBX-based strategy, we correlated the presence and/or absence of microbial transcripts with fecal N-acetyl 5-ASA levels across samples from 5-ASA users. Specifically, first we represented each metatranscriptomic gene family as present or absent, based on detection in the IBDMDB (relative abundance >0). Next, we classified stool samples as N-acetyl 5-ASA high (>median) or N-acetyl 5-ASA low/negative (<median, including undetectable levels). Then we calculated the sensitivity and specificity with which each metatranscriptomic gene cluster associated with the dichotomized N-acetyl 5-ASA. At a 50% cutoff for sensitivity and specificity (as used previously, Methods), we uncovered an additional 7 putative acetyltransferase gene clusters (FIG. 3b). One of these clusters that met our criteria overlapped with the GNAT N-acetyltransferase (C7H1G6) detected in our first step. Although they did not meet the sensitivity and specificity cutoffs used here, three of the acetyltransferase transcripts from the first criteria positively correlated with N-acetyl 5-ASA levels in users when considered as continuous values (p<0.05, FIG. 3c).

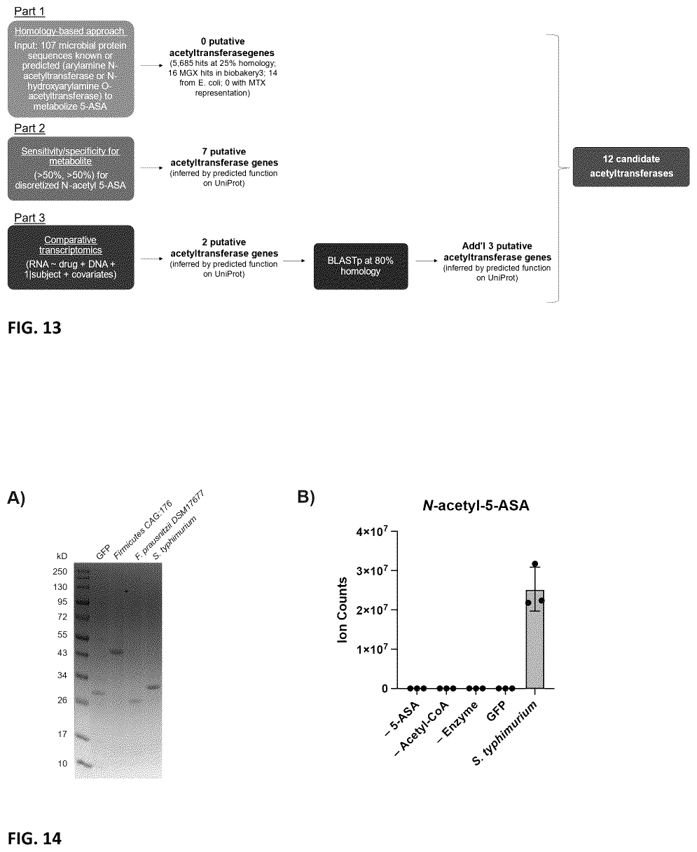

We finally pooled all candidate gene families that met either of these two MTX or MBX criteria (summarized in FIG. 13), yielding 12 candidate acetyltransferase gene clusters, which proved to bin (ClustalW, Methods) into two groups according to sequence similarity and membership in two large protein families: thiolases and acyl-CoA N-acyltransferases (FIG. 3d). The acyl-CoA N-acyltransferase family showed greater sequence heterogeneity (less strict conservation) than the thiolase family (mean alignment score 0.09±0.04 vs. 0.84±0.03) (FIG. 3e). The taxonomic origins of these protein families were also consistent. The acyl-CoA N-acyltransferase enzymes were almost all derived from the Bacteroidetes phylum, whereas the thiolase enzymes originated from the Firmicutes phylum, although many of these were from unclassified bacteria. One notable exception to this pattern was F. prausnitzii, which is a major member of the Firmicutes phylum. Exploring strain-level genomes, we found that only a subset of F. prausnitzii strains encoded the acetyltransferase of interest, and when hierarchically clustered according to a prior schema,³³ these appeared to originate from only one of the clade's major phylogroups, which could suggest possible horizontal gene transfer from a Bacteroidetes sp. to a common F. prausnitzii ancestor (FIG. 3f).”

### Microbial Thiolases Perform Promiscuous Acetylation of 5-ASA In Vitro

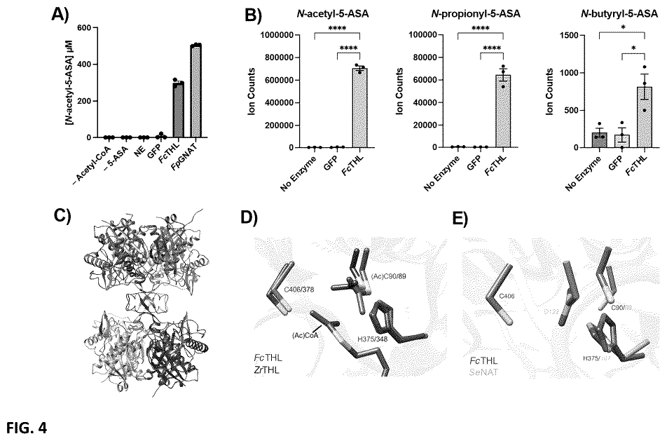

Having identified thiolase and acyl-CoA N-acetyltransferase superfamilies with potential 5-ASA-inactivating capabilities, we sought to biochemically confirm these predicted activities in vitro. Since examining the genomic context of each candidate using assembled complete genomes revealed no obviously related regulatory genes synthesized codon-optimized DNA sequences for each candidate gene according to their UniRef90 amino acid sequence and heterologously expressed them in E. coli(Methods). We expressed the known NAT from Salmonella enterica, the candidate thiolase from Firmicutes CAG:176 (UniRef90 ID R6CZ24), and the acyl-CoA N-acetyltransferase from F. prausnitzii (UniRef90 ID C7H1G6) for further biochemical characterization (FIG. 14a). An in vitro mass spectrometry assay for 5-ASA acetylation confirmed the ability of the Firmicutes CAG:176 thiolase and the F. prausnitzii acyl-CoA N-acetyltransferase to acetylate 5-ASA using acetyl-CoA, generating product at a level consistent with >25% conversion in a physiologic time frame (FIG. 4a). The known S. enterica enzyme served as a positive control (FIG. 14b).

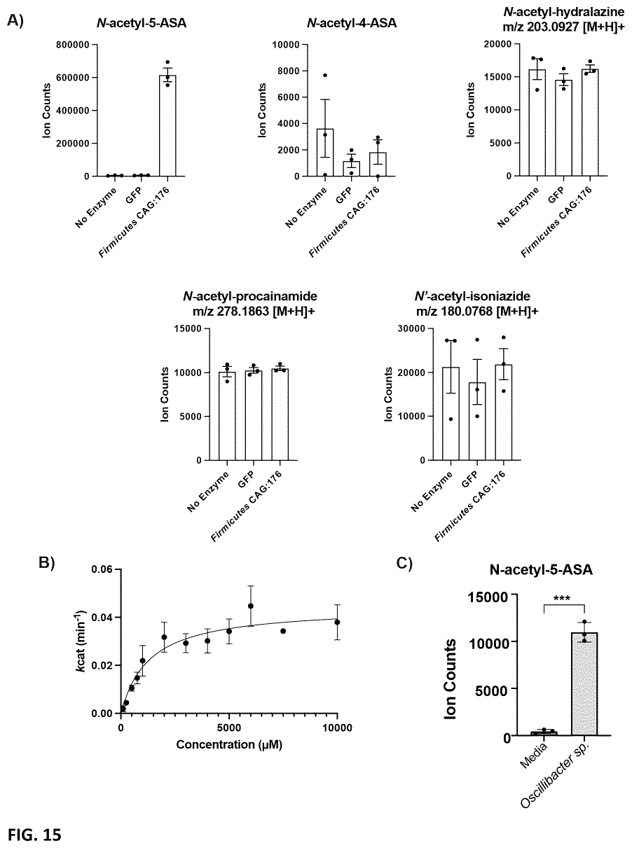

We focused our next efforts on the thiolase superfamily due to its combination of phylogenetic consistency and substantially greater sequence conservation. The thiolase enzyme also accepted longer chain acyl-CoA donors in a pooled assay (FIG. 4b), which supports our predicted assignment of the secondary biotransformation products of 5-ASA observed in patient samples (FIG. 2). We then tested the activity of the thiolase for acetylation of other xenobiotics containing amine groups, including the 5-ASA isomer, 4-ASA, and other common clinical compounds, such as isoniazid, procainamide, and hydralazine. In contrast to 5-ASA, we did not observe any acetylation of these compounds, which suggests some level of substrate selectivity (FIG. 15a). Kinetics analysis revealed a Km of 1.88 mM+/−0.46 mM, well below the observed intraluminal stool concentrations of 5-ASA (median of 30 mM)³⁴ and a kcat of 0.0647 min−1+/−0.0199, similar to that reported for the S. enterica NAT²⁰ (FIG. 15b). Finally, we noted that a live culture of Oscillibacter sp., strain KLE 1745 encoding a second predicted thiolase gene (R6TIX3) was also capable of acetylation of 5-ASA to N-acetyl 5-ASA (FIG. 15c).

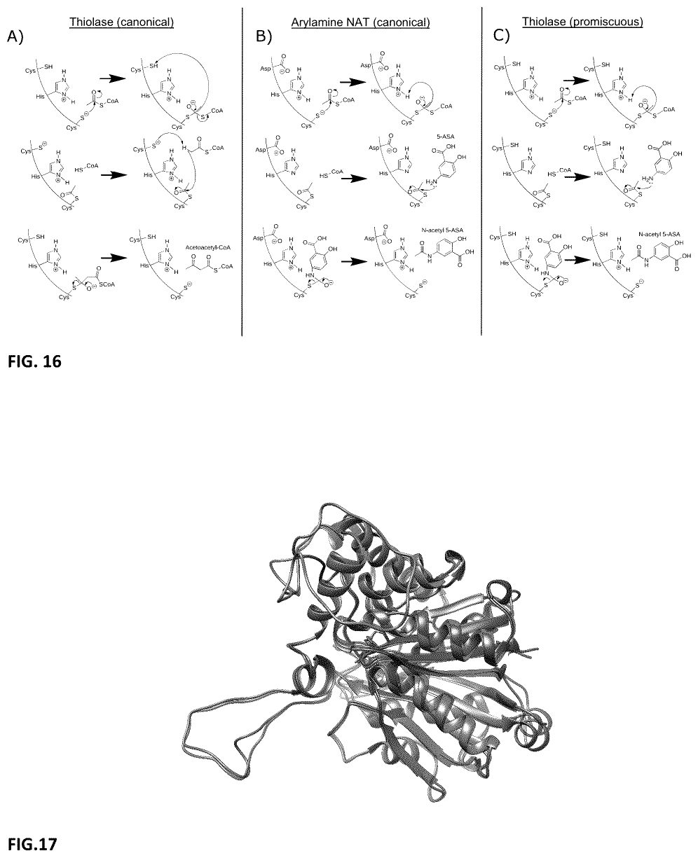

To gain insight into how the Firmicutes CAG:176 thiolase (FcTHL) acetylates 5-ASA, we generated an acetylated unliganded crystal structure of FcTHL refined at 1.9 Å resolution (Methods, Table 3). As seen in other biosynthetic thiolases,³⁵⁻³⁷ protein subunits join to form dimers, which then link via four interacting loops (one from each subunit) to form a tetramer (FIG. 4c). The four predicted active sites reside near these loops, projecting from the dimers, in the center of the tetramer.³⁵ To better understand the canonical thiolase mechanism, we then superimposed FcTHL on an acetylated thiolase crystal structure in complex with acetyl CoA from the well-characterized gram-negative Zoogloea ramigera (ZrTHL, PDB ID:1 DM3). ZrTHL is known to condense short-chain acyl-CoA molecules via a two-step “ping pong” mechanism (FIG. 16a) to generate acetoacetyl-CoA, used to synthesize polyhydroxybutyrate, a major bacterial energy storage molecule. Alignment of the two structures yielded a root mean square deviation (RMSD) of 1.652 Å (FIG. 17). The Cys-His-Cys triad in the known ZrTHL active site was conserved in FcTHL (FIG. 4d). In both enzymes, the first catalytic cysteine (ZrTHL:Cys89/FcTHL:90) is acetylated, forming a covalent acyl-enzyme intermediate, which is then poised to acetylate its substrate. In ZrTHL, when in complex with acetyl-CoA, this Cys89 rotates slightly to be parallel to the acetyl group to complete the condensation reaction. We next asked if this putative active site of the Firmicutes CAG:176 thiolase was similar to that of the S. enterica NAT, which is known to acetylate arylamines via a similar two-step “ping pong” mechanism (FIG. 16b). To do so, we aligned FcTHL with the crystal structure of the NAT (PDB ID: 2PFR). Intriguingly, the comparison also revealed acysteine and histidine dyad in the active site region (FIG. 4e).

Gut Microbial 5-ASA-Inactivating Acetyltransferases are Associated with Greater Risk of Treatment Failure in 5-ASA Users

Having identified human gut microbial acetyltransferases capable of converting 5-ASA to the clinically ineffective N-acetyl 5-ASA, we next examined whether the presence of these twelve enzymes was associated with 5-ASA treatment failure. Consistent with the primary outcome in prior clinical trials of 5-ASA, we defined this as initiation of corticosteroid treatments within our HMP2 patient subpopulation³⁸ (Methods). While the subcohort to which this was applicable was small, 39 individuals in the HMP2 who were treated with 5-ASA at any point and who provided longitudinal information on steroid use (prednisone, budesonide, methylprednisolone) contributed 609 stool samples across the entire year-long cohort.²² Using multivariate logistic regression models, we adjusted for age, sex, smoking status, and IBD subtype, each of which are thought to be linked to risk of disease flare.³⁹ We also were potentially able to account for host genetics, through inclusion of each participant's NAT2 phenotype (e.g. “fast” vs “slow” acetylator, Methods), as conflicting data suggest that NAT2 may or may not be implicated in 5-ASA metabolism.⁴⁰⁻⁴²

We found that the presence of any of four acetyltransferase genes in stool samples was significantly associated with an increased risk of steroid initiation (FIG. 5a)—three from the thiolase superfamily (R5CY66 OR 2.88, 95% CI, [1.66-5.00], and T5S060 OR 3.24, 95% CI [1.63-6.42]), including the Firmicutes CAG:176 enzyme characterized above (R6CZ24 OR 2.58, 95% CI [1.40-4.77]) and one from the acyl-CoA superfamily (C7H1G6, odds ratio [OR]2.81, 95% confidence interval [CI][1.68-4.68]). Carriage of the other eight candidate acetyltransferase genes identified above were not associated with an increased risk of steroid use (data not shown). As expected, earlier age of onset and CD vs UC status were similarly associated with greater risk of steroid initiation^{25,43} (Table 4). Importantly, we found no association between risk for steroid use and metagenomic presence of the E. coli NAT, identified from our initial homology search using the Salmonella NAT as a template sequence.

In sensitivity analyses, when considering each of these genes as part of a risk score, an increasing number of microbial acetyltransferase genes was significantly associated with a greater risk of steroid initiation (ptrend <0.0001), and models mutually adjusted for the other genes were essentially unchanged. Importantly, those with scores of ≥3 genes were similar in distribution by CD and age to those with 2 or fewer genes. When performing the same analyses among IBD participants who were never-users of 5-ASA (who instead were treated with other drugs e.g., on 6-MP, infliximab, or methotrexate), we found no positive association of these gene families with use of steroids. Further, we considered that participants' samples were not independent and thus we used mixed effects models adjusting for participant and found that presence of 3 or more acetyltransferase genes (compared to 2 or fewer) was also associated with an increased risk of steroid use, albeit with wider confidence intervals given the small sample size (OR 3.87, 95% CI 1.02-14.70) (FIG. 5b).

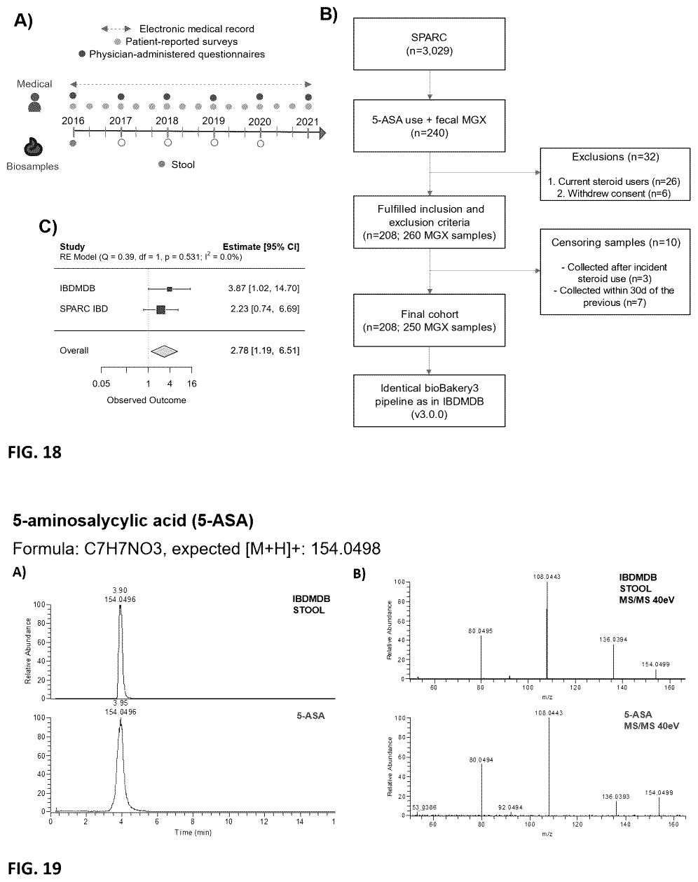

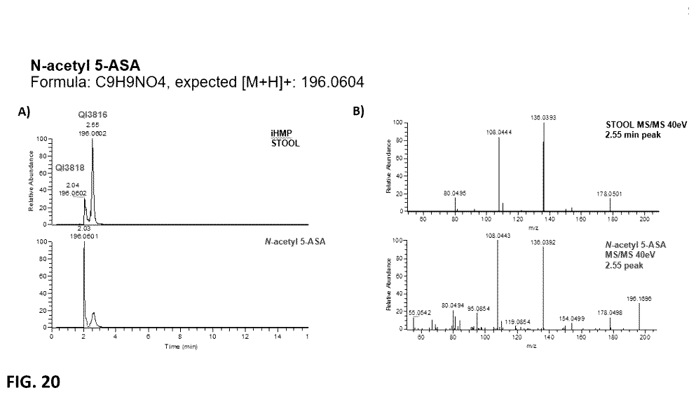

To test the reproducibility of our findings and to provide clarity about the temporal sequence between carriage of these microbial acetyltransferases and the transition from 5-ASA use to steroids, we validated our results in the independent Study of a Prospective Adult Research Cohort with IBD (SPARC IBD) (Methods). We note that we were blinded to these validation data, which were not available to us during the initial identification process for genes linked with steroid use in the IBDMDB. Among 208 users of 5-ASA who were steroid-free at study entry, we identified 60 new cases of corticosteroid use (FIG. 18, Table 5). Consistent with our analysis in the IBDMDB, we found that metagenomic carriage of 3 or more microbial acetyltransferase genes in SPARC IBD (compared to 2 or fewer) was associated with treatment failure (OR 2.77, 95% CI 1.03-7.43). In a pooled analysis of the IBDMDB and SPARC IBD results, the overall effect size suggested more than a three-fold increased risk of drug failure (OR 3.12, 95% CI 1.41-6.89) (FIG. 5b).

Finally, we observed that these acetyltransferase genes were variably prevalent across individuals, but also that their presence did not differ between 5-ASA users and non-users (FIG. 5c), supporting the notion that these genes are not merely a biomarker of medication status. Indeed, in further support of a microbiome-environment interaction—i.e. when microbial genes present at baseline become exposed to 5-ASA, they are overexpressed, which in turn leads to medication inactivation—we found overall similar prevalence rates of these enzymes among participants without IBD (Table 6).

### Discussion

Nearly forty years ago, researchers observed the conversion of 5-ASA to its inactive form (N-acetyl 5-ASA) in human stool cultures, leading to the hypothesis that gut bacteria may be responsible for this transformation.¹⁶ Fifteen years later, several bacteria, including S. typhimurium that contains a known homologue to the human NAT, were shown to inactivate 5-ASA.²⁰ However, the identity of the specific enzymes responsible for this conversion in healthy adults or in those with IBD remained unknown. Here, we offer evidence that the culprits are “moonlighting” gut commensal acetyltransferases⁴⁴—which canonically condense short chain acyl-CoAs as part of an intracellular energy storage strategy⁴⁵—and that they may have a direct, deleterious impact on host health.

Intriguingly, these findings highlight both the strengths and weaknesses of enzyme identification by sequence homology alone, as compared with the multi-omic methods we employed. While no transcribed gut microbiome sequences shared even remote homology with the known Salmonella NAT sequence, the predicted structural similarity between its active site residues and those of the Firmicutes thiolase could suggest an analogous and near-convergent enzymatic mechanism. Both enzymes form acyl-enzyme intermediates via cysteine residues that are poised to donate an acetyl group to a nucleophile, and both rely upon stabilizing histidine residues, providing a possible explanation (FIG. 16c) for the presumed off-target metabolism of 5-ASA by thiolases. Alternatively, the lack of concordance at the likely third active site residue could suggest a distinct mechanism either involving this second cysteine or facilitated by additional residues outside of the hypothesized active site. Further characterization, including crystallography with 5-ASA bound, will be necessary to unequivocally assess how these thiolases inactivate 5-ASA. It also remains to be seen if thiolase-mediated inactivation of 5-ASA confers any fitness advantage to microbial communities, or whether this is a truly off-target effect of the enzyme's natural activity.

More broadly, while some medications are known to influence the microbiome,⁴⁶ the converse effect of gut bacteria on drug safety and efficacy has even greater translational potential. One of the major challenges to discovering microbe-drug interactions is that their specificity can vary widely. In our study and recent case—examples,^{7,9-11} drug—metabolizing enzymes are restricted to very few microbial strains and sequences. Others, such as the microbial beta glucuronidases affecting cancer therapies,⁴⁷ can be taxonomically and phylogenetically widespread. In either case, such microbial enzymes are likely present in the host gut prior to any encounters with these non-antibiotic medications. Thus, the biomarker of interest for precision medicine is the baseline presence of relevant genes, which can often be identified by transcriptional responses to a compound, not the differential abundance of their microbial carriers. This necessitates novel and agnostic discovery methods such as those developed here—beginning with population-scale multi-omics (metatranscriptomics, metabolomics, and metagenomics) in the complex anaerobic nutritional and chemical environment of a natural human host—and ending with biochemical validation for the hundreds of health-relevant drug-microbe interactions that remain to be discovered.

Finally, our findings provide the first direct link between specific gut metabolic enzymes and 5-ASA treatment failure in IBD, thus yielding immediate clinical potential. 5-ASA is the most commonly prescribed medication for UC. To remain effective, it must remain available in unmodified form in the lumen of the colon, and thus, unlike other drugs, it is designed to be poorly absorbed in the small bowel.⁴⁸ Individuals in which 5-ASA therapy fails are typically progressed to riskier immunosuppressive treatments, and the ability to do so only when necessary (i.e. when 5-ASA-modifying thiolase sequences are present in the gut microbiome) would provide a valuable biomarker for precision medicine.

Furthermore, the elucidation of the enzymatic mechanisms by which microbes inactivate 5-ASA may lead to microbiome-specific inhibitors of enzymes to enhance 5-ASA efficacy in the future. In short, these findings open a conceptual window into personalized microbiome-based medicine for IBD.

The following materials and methods were used to obtain the above results.

### Materials and Methods

### Study Population and Biospecimen Sample Collection

As previously published,²² 132 IBDMDB participants were recruited between 2013-2014 from five major medical centers: Cincinnati Children's Hospital, Emory University Hospital, Massachusetts General Hospital, Massachusetts General Hospital for Children, and Cedars-Sinai Medical Center, and had an initial colonoscopy upon enrollment to determine study phenotype (UC, CD, or non-IBD). Based on a combination of endoscopic and histopathologic findings as well as gastrointestinal (GI) symptoms or positive imaging (for example, colonic wall thickening or ileal inflammation), 104 participants were classified as having UC or CD. Of these, 79 participants provided 1,036 stool samples every two weeks (either in person or by mail, deposited in 5 ml of molecular biology grade 100% ethanol, further processing details elsewhere²²) which were characterized by MGX, MBX, and/or MTX (see below). Due to restrictions such as available sample mass and missing samples, there were minor differences in the sampling pattern and in ability to perform MGX, MBX, and MTX on all samples. Blood draws (whole blood) occurred at approximately quarterly follow-up visits at the clinic.²² The study was reviewed by the Institutional Review Boards at each sampling site.²² All participants provided informed consent.

### Medication Assessment and Covariate Measurement

Throughout the study, participants completed serial questionnaires with information collected on diet, symptoms, and medication use.²² At baseline and with each collection of stool samples, participants completed a food frequency questionnaire, the former long-format and the latter short. Collection questionnaires also reported use of antibiotics, chemotherapy, or immunosuppressants such as steroids. More detailed medication information was collected three times at the three blood draws, at approximately the start, in the middle, and at the end of the study. Using this detailed data, we generated the following medication classes for mesalamine/5-ASA (oral: Asacol, Pentasa, Lialda, and Apriso; per-rectum: Rowasa enemas, Canasa suppositories; and bonded: Dipentum, Colazol, and Azulfidine). Steroids were grouped as use of Entecort, Medrol, or prednisone for the purposes of progression from 5-ASA in our case-cohort study (see ‘Case-cohort study below). Biologics were grouped as use of infliximab, adalimumab, certilizumab, and natalizumab. In the case of missing data, last observation carried forward (LOCF) was employed. Lifestyle and demographic data including age at consent, age at diagnosis, smoking status, and BMI were assessed at the start of the study. All questionnaires, as well as detailed protocols (including product numbers), can be found on the IBDMDB data portal at ibdmb.org/protocols. Responses and metadata are available at ibdmdb.org/results. Dysbiosis is a term previously defined²² as microbial excursions from the non-IBD microbiome that is considered to be both a surrogate of disease severity as well as activity.

As above, to maximize accuracy of 5-ASA user classification, use was determined according to detection of drug levels in stool (see section on ‘Metabolomics measurements and identification’). Based on two distinct groupings of 5-ASA levels in stool (FIG. 6), use of 5-ASA was defined biochemically, as containing levels of >107 in the IBDMDB MBX. Concordance was measured by statistical accuracy, according to the formula with TP as true positives and TN as true negatives:

(TP+TN)/(TP+TN+FP+FN).

In PRISM cohort metabolomics (FIG. 11), which was used as a validation cohort for discovery of unannotated compounds, given lower metabolomic abundance values overall due to normalization, use of 5-ASA was defined as levels >10⁵.

### Sequencing Assays

### DNA and RNA Isolation for Metagenomics and Metatranscriptomics

As previously described,²² total nucleic acid was extracted from an aliquot of each stool sample Chemagic DNA Blood Kit-96 from Perkin Elmer. This combines chemical and mechanical lysis with magnetic bead-based purification, with full details available from the IBDMDB. DNA samples were quantified using a fluorescence-based PicoGreen assay; RNA samples were quantified using a fluorescence-based RiboGreen assay (see below). RNA quality was assessed via smear analysis on the Caliper LabChip GX.

### Metagenome Sequencing

Metagenomes were generated as described elsewhere.²² In brief, metagenomic DNA was quantified using Quant-iT PicoGreen dsDNA Assay (Life Technologies) and normalized to a concentration of 50 pg/ul. Illumina sequencing libraries were prepared from 100-250 pg DNA using the Nextera XT DNA Library Preparation kit (Illumina). Prior to shotgun sequencing, libraries were pooled by collecting equal volumes of each library from batches of 96 samples. Insert sizes and concentrations for each pooled library were determined using an Agilent Bioanalyzer DNA 1000 kit (Agilent Technologies). Libraries were sequenced on HiSeq2000 or 2500 2×101 to yield ˜10 million paired end reads. Post-sequencing de-multiplexing and generation of BAM and FASTQ files were generated using the Picard suite (broadinstitute.github.io/picard).

### Metatranscriptome Sequencing

Illumina cDNA libraries were generated using a modified version of the RNAtag-seq protocol.⁴⁹ In brief, 500 ng-1 μg of total RNA was fragmented, depleted of genomic DNA, dephosphorylated, and ligated to DNA adapters carrying 5′-AN8-3′ barcodes of known sequence with a 5′ phosphate and a 3′ blocking group. Barcoded RNAs were pooled and depleted of rRNA using the RiboZero rRNA depletion kit (Epicentre). Pools of barcoded RNAs were converted to Illumina cDNA libraries and then sequenced as above.

### Exome Sequencing

Although only host NAT2 genotypes were used in this analysis (see below), whole-exome libraries were originally constructed and sequenced on an Illumina HiSeq 4000 sequencer with 151-bp paired end reads.²² Output from Illumina software was processed by the Picard pipeline to yield BAM files containing calibrated, aligned reads. Host genetic exome sequence data were processed using the Broad Institute sequencing pipeline by the Data Sciences Platform (Broad Institute).

### Metabolomics

### Stool Processing

A portion of each selected stool sample (40-100 mg) and the entire volume of originating ethanol preservative were stored in 15-ml centrifuge tubes at −80° C. until all samples were collected. Samples were then thawed on ice and centrifuged (4° C., 5,000 g) for 5 min. Ethanol was evaporated using a gentle stream of nitrogen gas using a nitrogen evaporator (TurboVap LV; Biotage) and stored at −80° C. until all samples in the study had been dried. After a homogenization and vortexing process, the mixture was aliquoted and stored at −80° C. until LC-MS analyses.

### Untargeted Assays

A combination of four untargeted LC-MS methods were used to profile metabolites in the fecal homogenates, as previously published;²² two methods that measure polar metabolites (a method that measures metabolites of intermediate polarity (for example, fatty acids and bile acids), and a lipid profiling method: 1) HILIC-pos (positive ion mode MS analyses of polar metabolites), 2) HILIC-neg (negative ion mode MS analysis of polar metabolites), 3) C18-neg (negative ion mode analysis of metabolites of intermediate polarity; for example, bile acids and free fatty acids), and 4) C8-pos. Lipids (polar and nonpolar). Additionally, pairs of pooled reference samples were inserted into the queue at intervals of approximately 20 samples for quality control and data standardization. Samples were prepared for each method using extraction procedures that are matched for use with the chromatography conditions. Data were acquired using LC-MS systems composed of Nexera X2 U-HPLC systems (Shimadzu Scientific Instruments) coupled to Q Exactive/Exactive Plus orbitrap mass spectrometers (Thermo Fisher Scientific).

### Taxonomic and Functional Profiling of Metagenomes and Metatranscriptomes

Taxonomic and functional profiles from the HMP2 were newly generated with the updated bioBakery3 meta'omics workflow using default parameters (huttenhower.sph.harvard.edu/biobakery_workflows) (20) (provenance log: ibdmdb.org/tunnel/cb/document/Public/HMP2/Metadata/anadama_run.log.gz). In brief, reads mapping to the human genome were first filtered out using KneadData v0.7.0. Taxonomic profiles of shotgun metagenomes were generated using MetaPhlAn 3.0, which uses a library of clade-specific markers to provide pan-microbial (bacterial, archaeal, viral, and eukaryotic) profiling (huttenhower.sph.harvard.edu/metaphlan3), quantifying a total of 578 microbial species across all participants (prior to quality control). Functional profiling was performed by HUMAnN v3.0.0.alpha.1 (huttenhower.sph.harvard.edu/humann3). HUMAnN constructs a sample-specific reference database from the pangenomes of the subset of species detected in the sample by MetaPhlAn (pangenomes are precomputed representations of the open reading frames of a given species). Sample reads are mapped against this database to quantify gene presence and abundance on a per-species basis. A translated search is then performed against a UniRef-based protein sequence catalog⁵⁰ (UniRef90 version 2019_01) for all reads that fail to map at the nucleotide level, in this case assigning 71.0%±13.3% of all DNA reads and 31.6%±8.8% of all RNA reads to UniRef90 gene families. The result are abundance profiles of gene families (UniRef90s), for both metagenomics and metatranscriptomics, stratified by each species contributing those genes, and which can then be summarized to higher-level gene groupings such as ECs or KOs. Relative abundance data was filtered to remove features with no variance or >90% zeroes and then arc-sine square-root transformed to reduce effects from zero-inflation. As reported previously,²² multiple pilot studies and technical replicates were performed as multi-batch analyses, ensuring that data generation methods produced reproducible results.

### Metabolomic Measurements and Identification

Raw LC-MS data were acquired to the data acquisition computer interfaced to each LC-MS system and then stored on a robust and redundant file storage system (Isilon Systems) accessed via the internal network at the Broad Institute. Nontargeted data were processed using Progenesis Qlsoftware (v 2.0, Nonlinear Dynamics) to detect and de-isotope peaks, perform chromatographic retention time alignment, and integrate peak areas. Peaks of unknown ID were tracked by method, m/z and retention time. Identification of nontargeted metabolite LC-MS peaks was conducted by: i) matching measured retention times and masses to mixtures of reference metabolites analyzed in each batch; and ii) matching an internal database of >600 compounds that have been characterized using the Broad Institute methods. Temporal drift was monitored and normalized with the intensities of features measured in the pooled reference samples.

For the identification of 5-ASA, two separate standards (SIGMA catalog PHR1060 and 18858) confirmed the identity of the 5-ASA peak in the IBDMDB (m/z: 154.0502, RT: 3.83 min) through retention time and spectral matching (FIG. 18).

### Statistical Analysis

### Impact of 5-ASA of the Fecal Metabolome and Microbial Contributions to 5-ASA Metabolism

To assess association of overall metabolome structure with 5-ASA use, omnibus testing was performed on Bray-Curtis dissimilarity matrices from MBX measurements. Profiles were first log-transformed and filtered to exclude features with more than 90% missing values before calculation of dissimilarities. Quantification of variance explained for the metabolomics data was calculated using PERMANOVA with the adonis function in the R v4.0.1 package “vegan” 2.5-6.⁵¹ The total variance explained by each variable was calculated independently of other variables to avoid issues related to ordering, as done previously.²² PCoA ordination in FIG. 2a was generated with the cmdscale function on Bray-Curtis distances, with unsupervised bivariate ellipses drawn at the 95% confidence level using the stat_ellipse function in the R package “ggplot2” v3.3.5.⁵²

To identify individual significantly differentially abundant metabolomic features before and after 5-ASA administration in FIG. 2b, we used paired two-sided Wilcoxon tests on data collected among 13 participants starting or resuming 5-ASA (“new users”), with an FDR threshold of 0.25. Box-plots elements shown here and throughout include medians as the center line, upper and lower quartiles as box limits; 1.5× interquartile range as whiskers; and outliers as points.

To identify possible biotransformations of 5-ASA, and specifically gain insights into the chemistry of microbial 5-ASA metabolism, we performed a four-step search, replicated in an independent cohort. First, we calculated mass differences (Δm/z) between the mass of each unannotated metabolite significantly altered by 5-ASA use (identified above, among the subpopulation of “new users”) and the expected parent compound (i.e. 5-ASA, a mass of 154.05). Second, we mapped these against known mass shifts from microbial biotransformations (6). Third, we calculated a c-statistic for each candidate metabolite, to determine how well it discriminated 5-ASA users from non-users in the entire study population, and subset these features to 95% predictive value (c-statistic >0.95). Finally, we further filtered this subset of annotated metabolomic features to those enriched among 5-ASA users compared to non-users (as opposed to enriched in non-users), given that drug derivatives should be more common among users. We then replicated this same four-step process in an independent cohort of 220 participants with and without IBD, the Prospective Registry in IBD Study at MGH (PRISM), which had fecal metabolomics generated using the same platform (26). The features from HMP2 and PRISM then were overlaid, yielding the proposed 5-ASA derivatives in FIG. 2c.

As further confirmation of our proposed derivatives, we pursued an independent metabolomics identification approach, where the known MS² spectra of N-acetyl 5-ASA (MassBank of North America) were passed to the GNPS workflow MASST,⁵³ then input to the METABOLOMICS SNETS-V2 workflow, and finally passed to a second GNPS workflow, Network Annotation Propagation (NAP_CCMS v1.2.5), all with default parameters.⁵⁴ The results were exported for visualization using the GNPS workflow MolNetEnhancer (v15). Nodes that were adjacent to (and thus co-occurring with) N-acetyl 5-ASA were inspected, and their m/z ratios were compared to the proposed candidates.

### Identification of Contribution of the Microbiome to 5-ASA Linked Metabolites

In order to identify relative contributions of microbial, host, and other factors to the subset of differentially abundant metabolomic features altered by 5-ASA in FIGS. 5a, we built linear regression models to predict log-transformed levels of each metabolite with four terms and an intercept. In an analysis restricted to 5-ASA users alone, these four terms comprised: 1) The “host data” feature group, comprising age, sex, disease type (CD, UC), use of antibiotics, and a Western dietary pattern (built from the per-sample food frequency questionnaires described above, where responses about dietary intake were converted to servings per day and separated into two well-established dietary patterns, a “Western” and “prudent”, using factor analysis with a varimax rotation as previously;⁵⁵ 2) The “drug data” group, represented by 5-ASA levels in stool; 3) The “microbiome data” group, including the first 10 principal components computed over the arc-sin square-root transformed relative abundance of taxonomic data as previously described;⁵⁶ and 4) An unknown/unexplained term. We then extracted the total variance explained by each model, and partitioned the relative contribution of each feature group to the overall R2, using the Lindemann, Merenda and Gold method in the R package “relaimpo” v2.2-3,⁵⁷ which considers the order of the sequence of predictors appearing in each model.

Differentially abundant microbial species between 5-ASA users and non-users were identified using arcsine square-root transformed taxonomic data with an FDR q<0.25. Abundances were fit with the following per-feature linear mixed-effects model:

$$species ∼ ( intercept ) + 5 - ASA use + diagnosis + antibiotiv use consent age + ( 1 / subject )$$

Using the significant results of this differential abundance testing among species and the significantly altered metabolites identified among new users, we then conducted, hierarchical all-against-all association testing using Spearman associations using HAIIA 0.8.20 for FIGS. 5b-d, with an FDR q threshold <0.05, (huttenhower.sph.harvard.edu/halla).

### Identification of Candidate Acetyltransferases

We first performed an amino acid homology search (blastp function, Diamond v0.9.24.125) of the Human Microbiome Project (HMP) reference isolate gut microbial genomes (UniRef90 version 2019_01) using an e-value of 10, >25% identity, “sensitive” mode, and 107 input microbial protein sequences (Table 4). Two came from arylamine N-acetyltransferases (NAT) from Salmonella enterica serovar typhimurium LT2 (nhoA) (UniProt accession, Q00267) and Pseudomonas aeruginosa (UniProt accession, Q9HUY3), previously shown to metabolize 5-ASA.^{20,21} 105 additional sequences were also included (that majority of which were NATs or N-hydroxyarylamine O-acetyltransferases), after having been generated by “Similarity to Identify MicrobioMe Enzymatic Reactions” (SIMMER),⁵⁸ a computational tool designed to make informed hypotheses about microbial enzymes predicted to metabolize xenobiotics, in part informed by prior experimental knowledge. For this tool, we provided the SMILES substrate input as:

C1=CC(═C(C═C1N)C(═O)O)O·CC(═O)SCCNC(═O)CCNC(═O)[COOH](C(C)(C)COP(═O)(O)OP(═O)(O)OC[COOH]1[COH]([COH]([COOH](O1)N2C═NC3═C(N═CN═C32)N)O)OP(═O)(O)O)O

And the SMILES product as:

CC(═O)NC1═CC(═C(C═C1)O)C(═O)O·CC(C)(COP(═O)(O)OP(═O)(O)OC[COOH]1[CO H]([COH]([COOH](O1)N2C═NC3═C(N═CN═C32)N)O)OP(═O)(O)O)[COH](C(═O)NCCC(═O)NCCS)O

We requested to report all alignments that were found and then overlapped the resultant list of 5,206 unique UniRef90 IDs with the entire catalog of UniRef90 IDs provided by the HMP2 MGX and MTX datasets.

In our MTX-based strategy, we used differential abundance testing to identify significantly overexpressed acetyltransferases based on paired MTX and MGX profiles. After an arcsine square-root transformation was applied to both MTX and MGX datasets, abundances were fit with the following per-feature linear mixed-effects model, which adjusted for DNA copy number, which allows for biological and technical zero values while also controlling for underlying DNA levels (27):

$$RNA ∼ ( intercept ) + 5 - ASA use + diagnosis + DNA + dysbiosis + antibiotic use consent age + ( 1 / subject )$$

as prior studies have shown that RNA levels can be a function of mere abundance of DNA.⁵⁹ Fitting was performed with the/me function from the R package “nlme” package v3.1-149,⁶⁰ where significance of the association was assessed using Wald's test. Nominal P values were adjusted for multiple hypothesis testing with a target FDR q of 0.25. In order to reduce the effect of zero inflation in microbiome data, features with a relative abundance of less than 1 e-8 in at least 10% of samples were excluded. Among the significant hits, we then sought to further characterize those with putative acyl transfer function, as defined by containing an acetyltransferase domain as annotated on UniProt.

In our MBX-based strategy, we dichotomized each metatranscriptomic gene family as present (relative abundance >0) or absent. Next, we classified stool samples as N-acetyl 5-ASA high (>median) or N-acetyl 5-ASA low/negative (<median, including undetectable levels). Then we calculated the sensitivity and specificity with which each metatranscriptomic gene cluster associated with the dichotomized N-acetyl 5-ASA. Filtering gene clusters with greater than 50% sensitivity and specificity (as used previously),⁶¹ we then sought to further characterize those with putative acyl transfer function, as defined by those containing an acetyltransferase domain as annotated on UniProt.⁶² For continuous correlations, abundance of the seven additional acetyltransferase gene clusters were then arc-sine square-root transformed and associated with log-transformed N-acetyl 5-ASA with linear regression, with an FDR q<0.25 threshold applied (95% CI shown in FIG. 3c).

The pooled twelve amino acid sequences were aligned with clustalW v2 (default parameters).⁶³ Candidate enzymes were then grouped in an average distance tree using neighbor-joining with Blosum62 (Jalview v2.11.1.3),⁶⁴ which were then mapped to the InterPro database 85.065 to identify protein superfamilies. Amino acid residues in the multiple sequence alignment were colored by clustalX v2.⁶³ Taxonomic lineages of each candidate enzyme was then determined using the UniProt database.⁶²

For gene context analysis, we downloaded the assembled reference genomes of the strains of interest from the NCBI assembly database⁶⁶ in July 2019. We then queried the proteins of interest (our candidate UniRef90s) against the proteins encoded by the reference genomes using DIAMOND v0.9.24,⁶⁷ and identified homologous loci by requiring identity 290% and coverage 280%. Next, we examined up to 10 genes that were upstream and downstream from these acetyltransferase gene loci to explore genomic neighborhoods. These neighborhood genes' UniRef accessions (from NCBI) were then mapped to the UniProt⁶² to assess their approximate functions, and in particular, were screened for any glosses indicating transcriptional regulators and/or promoters.

Finally, using previously defined phylogroups generated for F. prausnitzii,³³ based on a comparative analysis of genomes, in which a phylogenetic tree of the family Ruminococcaceae was constructed based on concatenated alignments of 245 highly conserved proteins. Within this hierarchy, F. prausnitzii was represented as a monophyletic group of strains, and within this, there were three clear and statistically significant splits into species/subspecies level groups (FIG. 3f). We explored all available F. prausnitzii genomes and annotated all strains containing and not containing the UniRef90 of interest.

### Case-Cohort Study

We examined the association of candidate acetyltranferases with risk of disease of relapse among a convenience sample of 39 “ever users” of 5-ASA—defined as any use of 5-ASA in the cohort. Participants with missing corticosteroid data were excluded. First, we discretized each of the 12 candidate protein families as present/absent based on MGX abundance per sample >0. We estimated multivariate odds ratios (ORs) for relapse with 95% confidence intervals (Cis) using logistic regression, adjusting for potential confounders, including smoking status (never vs ever), age at consent (continuous), disease type (CD vs UC), and host acetylation phenotype (defined below). We also tested for trend by considering each protein as an independent risk factor, and creating an additive score. In a sensitivity analyses, we tested the association between presence of the E. coli NAT identified in our homology search with steroid use, as well as the association between these acetyltransferases and risk of steroid use among 5-ASA non-users. Finally, as shown above in the results, our results were similar when we used fixed effects models and mixed effects models (including a random effects term for individual effects). In light of this similarity, and the fact that steroid use can truly occur multiple times throughout a year, we focused on the results of fixed effects models.

### Validation of Treatment Failure Based on Microbial Acetyltransferase Genes in SPARC IBD

### Data Source

We validated the association between metagenomic carriage of gut microbial acetyltransferases with risk of 5-ASA treatment failure in an ongoing independent cohort, Study of a Prospective Adult Research Cohort with IBD (SPARC IBD), from the IBD Plexus platform of the Crohn's & Colitis Foundation. As previously published,^{68,69} 3,029+well-phenotyped adult patients with UC, CD, or unclassified IBD have been recruited since 2015 from 17 major academic medical centers with no overlap with those in the IBDMDB: Baylor College of Medicine, Baylor University Medical Center, Brigham & Women's Hospital, Indiana University Hospital, Mayo Clinic, Medical College of Wisconsin, NYU Langone Medical Center, University of Alabama Medicine, University of Cincinnati Medical Center, University of Chicago, University of Maryland, University of Michigan, University of Pennsylvania, University of Pittsburgh, University of Wisconsin, Vanderbilt University Medical Center, Washington University School of Medicine. The study protocol was approved by the University of Pennsylvania's institutional review board. All participants provided informed consent.

### Study Population

The study population consisted of 240 patients enrolled in SPARC IBD between 2016 and 2020 who 1) provided a stool sample at time of consent and 2) were on 5-ASA at cohort entry. To ensure a prospective design, participants who were on steroids at baseline were excluded from this analysis (n=26). Additionally, we excluded participants who withdrew consent from the study after enrollment (n=6). Thus, the remaining 208 individuals formed the analytic sample. Follow up was through Oct. 19, 2021.

### Stool Sampling

At the time of consent, samples were collected by the patient at home and shipped directly to the biobank through a courier service as previously described.⁶⁹ A minority of individuals (n=33) provided more than one sample during the cohort. Although the SPARC IBD protocol does not include a prespecified schedule for repeated sampling after enrollment, participants are able to provide additional biosamples at time of usual care sigmoidoscopy or colonoscopy. Additional stool samples may also be obtained approximately 3 months after a change in therapy if the patient has a follow-up office visit during that time.

### Medication Data

At enrollment current medications were captured through the SPARC electronic data capture tool. Subsequent medication data in SPARC IBD were collected from an IBD Smartform and more broadly from the Epic electronic health record system. In addition, electronic surveys were delivered to patients every 3 months to capture IBD-related symptoms and current IBD therapies to track patient-reported disease activity between office visits and to further confirm medications. Use of 5-ASA was defined identically as in the IBDMDB by use any of the following, which includes bonded formulations: oral mesalamine, mesalamine suppository, mesalamine enema, olsalazine, balsalazide, and sulfasalazine. Use of corticosteroids was defined identically as in the IBDMDB by use any of the following: budesonide, prednisone, or methylprednisolone.

### DNA Extraction

Genomic DNA extraction was performed via the Mag Attract Power Soil kit (Qiagen, Cat #27000-4-EP) following the manufacturer's instructions. Total nucleic acids were extracted using the Qiagen MagAttract PowerMicrobiome kit (Qiagen, Catalog No. 27500-4-EP).

### Metaqenomic Sequencing

Metagenomic sequencing was performed by Diversigen. Genomic DNA was prepared into libraries for sequencing by the Nextera DNA Flex library preparation kit (Illumina, Catalog No. 20018705) with Nextera Index Kit (Illumina, Catalog No. 20027213). Library size estimation and quantification were determined with the fragment analyzer (Advanced Analytical Technologies, Inc.) electrophoresis system. The prepared libraries were sequenced via the NovaSeq 6000, 2×150 bp sequencing platform (Illumina).

### Statistical Analysis

Data from the validation cohorts were not available to us while developing the original prediction models in the HMP2. Sequencing data from SPARC IBD went through the exact same analysis pipeline (biobakery3) as in the IBDMDB to extract the metagenomic data that our prediction models were based on. Only microbial acetyltransferases that were significantly predicted to be linked with steroid use in our discovery cohort (R6CZ24, T5S060, R5CY66, C7H1G6) were considered for further analysis. As in the IBDMDB, we discretized each of the four candidate protein families as present/absent based on abundance per sample >0. To mitigate sampling bias, samples were not included if they were provided fewer than 30 days after the previous. Similarly, to avoid sampling bias, participants were censored when they reported using steroids.

The primary exposure was defined as metagenomic carriage of 3-4 acetyltransferases compared to 0-2 acetyltransferases, just as performed in the IBDMDB. We were not powered to examine the association between each acetyltransferase with risk of steroids. Given the possibility of correlation within samples, we calculated the odds ratios (ORs) and 95% confidence intervals (Cis) of use of steroids using multivariable generalized estimating equations in the in the R v4.0.1 package “gee” v. 4.13⁷⁰ with adjustment for age and sex. In a sensitivity analysis, we limited our analysis to the single baseline sample, and related metagenomic carriage of these genes with future steroid risk using multivariable logistic regression with adjustment for age, sex, and IBD disease type. Random effects meta-analysis was performed using the R v4.0.1 package “metafor” 1.4-0.⁷¹

### Assignment of Host Acetylation Phenotype

Human NAT2 acetylator genotype status has previously been linked with increased risk of bladder cancer due to decreased ability to detoxify carcinogens,⁷² as well in predicting drug-induced liver injury from isoniazid.⁷³ Although studies attempting to link NAT2 acetylation phenotypes with 5-ASA response have not been fruitful,⁴⁰ many continue to speculate if NAT2 could play a role in inactivating 5-ASA.⁴¹ Therefore, using a simple panel of two SNPs identified in participants' exome sequencing data (rs1041983, rs1801280) previously shown to accurately predict acetylation phenotypes as “fast” vs. “slow”,⁷⁴ we first calculated the sum of variant alleles in each patient. As previously, any participant who had two or more variant alleles could then be categorized as a slow acetylators, inferring that they had either the NAT2*5 or NAT2*6 haplotype.

### Expression, Purification and In Vitro Assessment of Candidate Acetyltransferase Enzymes

Candidate 5-ASA acetyltransferase genes (as well as control GFP and known Salmonella typhymirium gene) were synthesized according to the E. coli codon-optimized amino acid sequence on UniProt (GenScript) and then cloned into pET28b inducible expression vectors using Gibson assembly (including an in-frame either N- or C-terminal polyhistidine sequence). Identities of the constructs were confirmed with DNA sequencing and then were transformed into E. coli BL21 strains for expression according to the standardized protocols from GenScript. All E. coli expression constructs were grown anaerobically in sealed Hungate tubes in Terrific Broth (VWR) supplemented with 5 mM MgSO4 and kanamycin (50 μg/mL) and were incubated at 37° C. overnight before dilution at 1:100 into fresh media the following morning. These diluted cultures were grown anaerobically in sealed flasks at 37° C. to an OD600 ˜0.6 at which point protein expression was induced by the addition of 100 μM Isopropyl β-D-1-thiogalactopyranoside (IPTG, TEKNOVA) followed by aerobic culture overnight at 16° C. The following morning, E. coli were pelleted by centrifugation and then lysed in 20 mM HEPES pH 8.0 buffer containing 30 mM imidazole and 300 mM NaCl and supplemented with 0.5% Octyl-β-D-thrioglucopyranoside (Chem-Impex), 0.5 mg/mL lysozyme (Sigma), and SIGFAST protease inhibitor cocktail (Sigma). After lysis and clarification by centrifugation, lysates were incubated for 1 hour at 4° C. with His-Pure Cobalt Purification beads (Thermo). After incubation, beads were washed with 6 column volumes of 20 mM HEPES pH 8.0 buffer containing 30 mM imidazole and 300 mM NaCl before elution with 1 column volume of 20 mM HEPES pH 8.0 containing 300 mM imidazole and 300 mM NaCl. Eluted protein was then buffer exchanged into 20 mM HEPES pH 8.0 with 300 mM NaCl and 10% glycerol for storage at −80° C. prior to further experiments.

Enzyme concentrations were calculated according to Beer's Law with extinction coefficients calculated by Benchling software based on the amino acid sequences of the putative enzymes. Assays mixtures contained 20 mM HEPES pH 7.5 with 50 mM NaCl with 50 μM enzyme for the end-point assay and 5 μM for promiscuity assays; 1 mM of the specified substrates (5-ASA, 4-ASA, dapsone, isoniazid, procainamide, hydralazine), and 1 mM of the indicated acyl-CoAs (acetyl-CoA, propionyl-CoA, butyryl-CoA, CoALA Biosciences) supplemented with 1 mM MgCl₂ and 1 mM Tris-(2-carboxyethyl)phosphine (TCEP, Sigma). Reactions were conducted at 37° C. for 6 hours for the initial endpoint assay and room temperature for 1 hour for the promiscuity assay. Quenching/extraction for the promiscuity assay was with equal volumes of acetonitrile and methanol. Quenching/extraction for the end-point was one part sample and nine-parts extraction mix (75% acetonitrile:25% methanol v/v with 10 μM 1,2-¹³C₂-taurine from Cambridge Isotopes as an internal standard for quantification). Samples were vortexed and cooled to −20° C. and then centrifuged prior to LC-MS analysis.

For the kinetic assay, assay conditions were the same as above (20 mM HEPES pH 7.5 with 50 mM NaCl, 1 mM MgCl₂, and 1 mM TCEP, and 5 μM enzyme). Concentration of 5-ASA substrate was varied as indicated with 1 mM acetyl-CoA held constant. Reactions were carried out at 37° C. for 0, 10, 20, 30, 45, 60, 90, and 120 min in triplicate and quenched/extracted with one part sample and nine-parts extraction mix (75% acetonitrile: 25% methanol v/v with 10 μM 1,2-¹³C₂-taurine from Cambridge Isotopes as an internal standard for quantification). Samples were vortexed and cooled to −20° C. and then centrifuged prior to LC-MS analysis.

For the promiscuity assays, mass spectrometry analyses were conducted using an LC-MS system composed of an Agilent 1260 Infinity HPLC (Agilent Technologies) coupled to a 6530 Accurate-Mass QTOF LC-MS (Agilent Technologies). The samples were injected into a 150×3 mm Hypersil GOLD aQ column (Thermo Scientific) at 30° C. The column was eluted isocratically at a flow rate of 500 μL/min with 99% mobile phase A (0.1% formic acid in water) for two minutes followed by a linear gradient for 16 minutes to 99% mobile phase B (acetonitrile with 0.1% formic acid). This ratio was continued for 5 minutes before returning to 99% mobile phase A over 1 minute and continuing at that ratio for additional 6 minutes. MS analyses were performed in negative ion mode (N-acetyl-5-ASA, N-propionyl-5-ASA, N-butyryl-5-ASA, and N-acetyl-4-ASA) and positive ion mode (N-acetyldapsone, N-acetylprocainamide, N-acetylhydralazine, and N′-acetylisoniazid) using electrospray ionization, full scan MS acquisition over 100 to 3000 m/z, a resolution setting of 10,000, and centroided masses. Other MS settings were as follows: heater temperature 300° C., ESI nebulizer 35 psi, spray voltage 3.5 kV, 2 spectra/s. The identities of N-acetyl-5-ASA (Cayman Chemical, Ann Arbor, MI) and N-acetyl-4-ASA (Santa Cruz Biotechnology, Dallas, TX), were confirmed using authentic mass standards. Remaining identities were inferred using calculated masses with a mass detection tolerance of 10 ppm. Raw data from the LC-MS were analyzed using Agilent MassHunter Qualitative Analysis 10.0 software.

For end-point and kinetic assays, mass spectrometry analyses were conducted using an LC-MS system composed of an Agilent 1290 Infinity II UHPLC (capable of column switching) coupled to a Agilent 6470A Triple Quadrupole LC/MS. The samples were injected into an Infinity Lab Poroshell120 Hilic column (2.1×100 mm 2.7 μm) at 25° C. The column was eluted isocratically at a flow rate of 600 μL/min with 5% mobile phase A (10 mM ammonium formate with 0.1% formic acid in water) for 18 seconds followed by a linear gradient for 132 seconds to 60% mobile phase B (acetonitrile with 0.1% formic acid). This was followed by a 3 second gradient to 40% mobile phase B. This was then followed by a 27 second gradient returning to 5% mobile phase A at a flow rate of 1200 μL/min. This flow rate and ratio was held for an additional 48 seconds. Additional column equilibration was carried out on a secondary pump for 117 seconds at 5% mobile phase A and a flow rate of 1000 μL/min. MS was conducted in negative ion mode using electrospray ionization. Data were collected via MRM (N-acetyl 5-ASA MRM 194->150, 1,2-¹³C₂-taurine MRM 125.9->79.9). Other MS settings were as follows: heater temp 300° C., ESI nebulizer 45 psi, spray voltage 3.5 kV, acquisition time 100 ms/spectrum. Raw data from the LC-MS were analyzed using Agilent MassHunter Quantitative Analysis Version 10.1 Software. For absolute quantification, all samples were normalized to the taurine internal standard and concentrations calculated against a standard curve.

### Oscillibacter Culture

Oscillibacter sp., strain KLE 1745 was obtained through BEI resources, the NIH Institute of Allergy and Infectious Diseases (NIAID) as part of the Human Microbiome Project. This strain was cultured anaerobically directly from freezer stocks for three days in reinforced Clostridial media (BD Difco) at 37° C. before the addition of 5-ASA to a final concentration of 1 mM (stock concentration 100 mM in DMSO, sterile filtered with 0.22 μm filter) for an additional 24 hours prior to harvesting. Spent media was centrifuged at 10,000×g for 3 minutes to pellet bacteria and cell debris. 10 μL of media was then extracted with 90 μL of acetonitrile:methanol (75%:25% v/v) and measured via LC-QTOF as described elsewhere in the methods section.

### Crystal Structure Analysis

### Protein Expression and Purification

The C-terminal non-cleavable His tag construct of the thiolase from the Firmicutes CAG:176 thiolase (FcTHL) was overexpressed in E. coli BL21 (DE3) and purified using affinity chromatography and size-exclusion chromatography. Briefly, cells were grown at 37° C. in TB medium in the presence of 50 μg/ml of kanamycin to an OD of 0.8, cooled to 17° C., induced with 500 μM isopropyl-1-thio-D-galactopyranoside (IPTG), incubated overnight at 17° C., collected by centrifugation, and stored at −80° C. Cell pellets were lysed in buffer A (25 mM HEPES, pH 7.5, 500 mM NaCl, 0.5 mM TCEP, and 20 mM Imidazole) using Microfluidizer (Microfluidics), and the resulting lysate was centrifuged at 30,000 g for 40 min. Ni-NTA beads (Qiagen) were mixed with cleared lysate for 30 min and washed with buffer A. Beads were transferred to an FPLC-compatible column, and the bound protein was washed further with buffer A for 10 column volumes and eluted with buffer B (25 mM HEPES, pH 7.5, 500 mM NaCl, 0.5 mM TCEP, and 400 mM Imidazole). The eluted sample was concentrated and purified further using a Superdex 200 16/600 column (Cytiva) in buffer C containing 20 mM HEPES, pH 7.5, 200 mM NaCl, and 10 μM TCEP. FcTHL containing fractions were concentrated to ˜50 mg/mL and stored in −80° C.

### Crystallization

FcTHL at 800 μM was crystallized in 100 mM sodium acetate, pH 4.9, 55% MPD, and 20 mM CaCl₂) by sitting-drop vapor diffusion at 20° C. Crystals were transferred briefly into crystallization buffer containing 25% glycerol prior to flash-freezing in liquid nitrogen.

### Determination

Diffraction data were collected at beamline NE-CAT-241D-E at the Advanced Photon Source (Argonne National Laboratory). Data sets were integrated and scaled using XDS.⁷⁵ Structures were solved by molecular replacement using the program Phaser and the search model PDB entry 4XL2. Iterative manual model building and refinement using Phenix⁷⁶ and Coot⁷⁷ led to a model with excellent statistics.

### Visualization

Protein structures were visualized in ChimeraX v1.15.⁷⁸ The predicted structure was then overlaid using the MatchMakertool (default settings) on monomers from the PDB entries 1 DM3 and 1 E2T. To compare active sites, residues were pre-specified, and then overlaid using the match tool(default settings) which performs least-squares fitting of active site atoms, first moving the one set of atoms onto the second, followed by the remaining model containing the atoms.

TABLE 1
Characteristics of 5-ASA users vs non-users in the IBDMDB. IBD
users of 5-ASA were more likely to have ulcerative colitis.
Overall, rates of hospitalization and history of bowel surgery
were also lower for 5-ASA users compared to non-users.

| | Non-users | Users | |
|---|---|---|---|
| | (n = 34) | (n = 45) | p-value |
| Age at consent, mean (SD) | 27.7 (17.4) | 26.7 (17.1) | 0.81 |
| Age at diagnosis, mean (SD) | 22.1 (14.2) | 20.9 (11.0) | 0.68 |
| Male (%) | 17 (50) | 21 (47) | 0.95 |
| UC (%) | 6 (18) | 24 (53) | 0.003 |
| Race/ethnicity (%) | | | |
| White | 30 (88) | 40 (89) | 0.13 |
| Other | 4 (12) | | 5 (11) |
| Prior bowel surgery (%) | | | 0.11 |
| Yes | 7 (21) | | 4 (9) |
| Antibiotic use (%) | 14 (41) | 25 (56) | 0.30 |
| Bonded 5-ASA use (%)^{a} | 0 (0) | 4 (9) | 0.20 |
| Hospitalized during the study (%) | 13 (38) | 7 (15) | 0.04 |
| Dysbiosis^{b} | 21 (62) | 19 (42) | 0.14 |

^{a}Bonded 5-ASA formulations include sulfasalazine, balsalazide, and olsalazine
^{b}As previously defined in the HMP2, Methods²²

TABLE 2
M/z ratios and retention times for 5-ASA in the IBDMDB and
PRISM cohorts. Identity of N-acetyl 5-ASA in PRISM and for
N-propionyl 5-ASA and N- butyryl 5-ASA in the IBDMDB and PRISM
cohorts were inferred using mass and retention time matching coupled
with known mass shifts from functional groups. As further confirmation
of these two possible 5-ASA derivatives, we searched publicly available
MS² data from the Global Natural Product Social (GNPS) database
(MassIVE MSV000084556) to generate molecular co-occurrence
networks, inputting the known spectra of N-acetyl 5-ASA (46).
In this analysis, we similarly found two unlabeled nodes linked
with N-acetyl 5-ASA which matched the m/z of the compounds
we identified, as well as a third node with an m/z of 238.11
which may correspond to yet another derivative of 5-ASA that
has not been described, likely a 5-carbon acyl group plus
5-ASA given the additional mass shift (+12) from butyryl
CoA.

| | PRISM | | IBDMDB | |
|---|---|---|---|---|
| | | Retention | | Retention |
| Compound | M/Z | time | M/Z | time |
| 5-ASA | 154.0493 | 3.98 | 154.0502 | 3.83 |
| N-butyryl 5-ASA | 224.0909 | 2.32 | 224.0922 | 2.24 |
| N-acetyl 5-ASA | 196.0599 | 2.97 | 196.0609 | 2.81 |
| N-propionyl 5-ASA | 210.0754 | 2.32 | 210.0764 | 2.52 |

TABLE 3
Data processing refinement statistics
and quality of the FcTHL model.

| | FcTHL |
|---|---|
| nan | ACAT - - -, R6C224 - - -, IFFNDC |
| Wavelength | 0.9792 |
| Resolution range | 73.46-1.89 (1.958-1.89) |
| Space group | P 1 |
| Unit cell | 75.23 81.46 160.19 95.61 100.45 108.27 |
| Total reflections | 500387 (50866) |
| Unique reflections | 263887 (26397) |
| Multiplicity | 1.9 (1.8) |
| Completeness (%) | 94.02 (93.71) |
| Mean I/sigma(I) | 7.10 (0.81) |
| Wilson B-factor | 39.07 |
| R-merge | 0.0697 (1.012) |
| R-meas | 0.09856 (1.432) |
| R-plm | 0.0697 (1.012) |
| CC1/2 | 0.99 (0.391) |
| CC* | 0.997 (0.75) |
| Reflections used in | 263709 (26331) |
| refinement | |
| Reflections used for | 13240 (1334) |
| R-free | |
| R-work | 0.1765 (0.3742) |
| R-free | 0.2079 (0.3960) |
| CC(work) | 0.964 (0.694) |
| CC(free) | 0.951 (0.643) |
| Number of non-hydrogen | 25877 |
| atoms | |
| macromolecules | 24521 |
| ligands | 0 |
| solvent | 1356 |
| Protein residues | 3364 |
| Nucleic acid bases | nan |
| RMS(bonds) | 0.010 |
| RMS(angles) | 1.05 |
| Ramachandran favored (%) | 96.84 |
| Ramachandran allowed (%) | 3.13 |
| Ramachandran outliers (%) | 0.03 |
| Rotamer outliers (%) | 0.96 |
| Clashscore | 4.25 |
| Average B-factor | 50.84 |
| macromolecules | 50.84 |
| ligands | nan |
| solvent | 50.74 |
| Number of TLS groups | 34 |

TABLE 4
Presence of 5-ASA metabolizing acetyltransferases are associated
with steroid use in the HMP2. Odds ratios with 95% CI are presented.
Analyses were adjusted for age, sex, disease type (CD vs. UC),
host acetylation phenotype, and smoking status.

| Feature | | |
|---|---|---|
| Genes | Absent | Present |
| C7H1G6 | 1 (referent) | 2.81 (1.68-4.68) |
| R5CY66 | 1 (referent) | 2.88 (1.66-5.00) |
| R6CZ24 | 1 (referent) | 2.58 (1.40-4.77) |
| T5S060 | 1 (referent) | 3.24 (1.63-6.42) |

| Host factors | | |
|---|---|---|
| Disease type | UC | CD |
| | 1 (referent) | 3.11 (1.85-5.21) |
| Age, y | 0.92 (0.89-0.94) | |

TABLE 5
Characteristics of the study population selected from the SPARC
IBD cohort. Compared to the IBDMDB, given that SPARC IBD is
a cohort of adult patients, participants were older. They
were also more likely to have UC and were less likely to be
male. The prevalence of acetyltransferases that were linked
with treatment failure was similar in both cohorts.

| | Users |
|---|---|
| | (n = 208) |
| Age at consent, mean (SD) | 45.5 (15.1) |
| Male (%) | 88 (42.3%) |
| IBD^{a} subtype | |
| UC (%) | 140 (67.3) |
| CD (%) | 67 (32.2) |
| IBD-U a (%) | 1 (0.4) |
| Prevalence of acetyltransferases, % | |
| C7H1G6 | 44.0 |
| R5CY66 | 20.4 |
| R6CZ24 | 7.2 |
| T5S060 | 18.8 |

^{a}IBD-undifferentiated

TABLE 6
Presence of 5-ASA inactivating acetyltransferases. Gene prevalence
(%) varies across participants with IBD, but does not vary
significantly by 5- ASA status (GLMM p > 0.07), arguing
for effect modification of the effects of 5-ASA on prevention
of disease relapse. Among participants without IBD prevalence
rates of these enzymes also appear similar.

| UniRef90 ID | 5-ASA users | 5-ASA nonusers | Non-IBD |
|---|---|---|---|
| C7H1G6 | 47.8 | 52.3 | 67.0 |
| R5CY66 | 17.4 | 20.3 | 43.7 |
| R6CZ24 | 20.7 | 14.3 | 28.5 |
| T5S060 | 14.1 | 9.2 | 12.3 |

### Use

We identified and biochemically validated gut microbial enzymes capable of generating the clinically ineffective 5-ASA derivative, N-acetyl 5-ASA, and we have shown that the presence of these microbial acetyltransferase genes in gut metagenomes is associated with an increased risk for 5-ASA treatment failure. These findings were driven and validated by metagenomics (MGX), metatranscriptomics (MTX), and metabolomics (MBX) from >1,000 stool samples collected over a one-year period from patients with IBD and controls,²² as well as subsequent phylogenetics, heterologous expression, and chemical characterization of the target proteins (which would otherwise have been annotated to a sequence superfamily canonically involved in energy storage). The process is generalizable to other endpoints, chemicals, and microbiome multi-omics, improving our ability to understand gut microbial metabolism of drugs in patients and, therefore, opening new avenues for targeted or adjuvant therapies.

These gut microbial enzymes are human gut microbial acetyltransferases capable of converting 5-ASA to the clinically ineffective N-acetyl 5-ASA. The presence of any of four acetyltransferase genes in stool samples was significantly associated with an increased risk of steroid initiation—three from the thiolase superfamily (R5CY66 OR 2.88, 95% CI, [1.66-5.00]; and T5S060 OR 3.24, 95% CI [1.63-6.42]), including the Firmicutes CAG:176 enzyme (R6CZ24 OR 2.58, 95% CI [1.40-4.77]) and one from the acyl-CoA superfamily (C7H1G6, odds ratio [OR]2.81, 95% confidence interval [CI][1.68-4.68]). Accordingly, a method of determining if a patient diagnosed with IBD will respond to 5-ASA therapy is provided. The method includes detecting in the patient gut bacteria having three or more of these genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii

The presence of three or more of these genes indicates that the patient diagnosed with IBD will not respond 5-ASA therapy and other therapies should be administered, for example: biologics (e.g. infliximab, adalimumab, vedolizumab) or immunomodulators (6-mercaptopurine, tofacitinib). If three or more of the above enumerated genes are not detected in the patient diagnosed with IBD, then the patient should be administered 5-ASA as a first line treatment according to standard treatment protocols.

The invention therefore includes a method of determining whether a patient diagnosed with IBD will respond to 5-ASA therapy. In this method a stool sample is obtained from the patient. The stool sample is then analyzed for the presence of microbial acetyltransferase genes capable of converting 5-ASA to the clinically ineffective N-acetyl 5-ASA. If microbial acetyltransferase genes are not present in the sample, the patient will respond to 5-ASA therapy and such therapy should be administered. However, if microbial acetyltransferase genes are present in the sample, the patient will not respond to 5-ASA therapy and a second line therapy should be administered. The microbial acetyltransferase genes are three or more of the following genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii.

The amino acid sequence and the GenBank Accession numbers for each of the four microbial acetyltransferase can be found here:
Firmicutes bacterium CAG:176: See www.uniprot.org/uniprotkb/R6CZ24/entry
Ruminococcaceae bacterium D16 See www.uniprot.org/uniprotkb/T5S060/entry
Firmicutes bacterium CAG:83: See www.uniprot.org/uniprotkb/R5CY66/entry
Faecalibacterium prausnitzii: See www.uniprot.org/uniprotkb/C7H1G6/entry

The nucleotide sequences can also be accessed using links on the uniprot.org site.

The method can be used if the IBD diagnosis is either ulcerative colitis or Crohn's Disease. The second line therapy can be selected from biologics (e.g. adalimumab, vedolizumab, infliximab) or immunomodulators (e.g. 6-mercaptopurine, tofacitinib) used for treating these diseases and such treatments are administered according to standard methods.

The detection of these microbial genes can be accomplished through readily used assays including shotgun sequencing (metagenomics) of human stool samples with computational analysis to identify the presence or absence of the relevant bacterial genes. The presence of these microbial genes at baseline predicts future loss of response to 5-ASA.

In other methods, qPCR (quantitative polymerase chain reaction) can be employed to identify the presence or absence of the relevant bacterial genes according to standard methods. Primer 3 software, which can be accessed at primer3.org/, is employed to design primers highly specific to the enzymes coded for by the above-identified genes. Those primers are then validated in silico. Either GeneRunner or BLAST (or both) can be used to check and confirm their efficiency and specificity. Then the in silico primers are further validated experimentally. See, for example, Kumar and Chordia, (2015) In Silico PCR Primer Designing and Validation, in Basu, C (eds) PCR Primer Design. Methods in Molecular Biology, vol 1275. Humana Press, New York, NY. Once validated, they are used to amplify the four genes set forth above according to Taqman fluorogenic probe detection. This can be accomplished by one of skill in the art using standard methods known in the art. See also, Barghouthi, A Universal Method for the Identification of Bacteria Based on General PCR Primers, Indian J Microbiol (October-December 2011) 51(4): 430-444 and Kralik and Ricchi, A Basic Guide to Real Time PCR in Microbial Diagnostics: Definitions Parameters, and Everything, Frontiers in Microbiology, 2 Feb. 2017, Vol 8, Article 108, doi: 10.3389/fmicb.2017.00108.

### REFERENCES

1. Plichta, D. R., Graham, D. B., Subramanian, S. & Xavier, R. J. Therapeutic Opportunities in Inflammatory Bowel Disease: Mechanistic Dissection of Host-Microbiome Relationships. Cell 178, 1041-1056 (2019).
2. Ham, M. & Moss, A. C. Mesalamine in the treatment and maintenance of remission of ulcerative colitis. Expert Rev Clin Pharmacol 5, 113-123 (2012).
3. Klag, T., Stange, E. F. & Wehkamp, J. Management of Crohn's disease—are guidelines transferred to clinical practice?United European Gastroenterology Journal 3, 371-380 (2015).
4. Ford, A. C. et al. Efficacy of 5-Aminosalicylates in Ulcerative Colitis: Systematic Review and Meta-Analysis. Official journal of the American College of Gastroenterology/ACG 106, 601-616 (2011).
5. Ford, A. C. et al. Efficacy of 5-Aminosalicylates in Crohn's Disease: Systematic Review and Meta-Analysis. Official journal of the American College of Gastroenterology/ACG 106, 617-629 (2011).
6. Javdan, B. et al. Personalized Mapping of Drug Metabolism by the Human Gut Microbiome. Cell 181, 1661-1679.e22 (2020).
7. Zimmermann, M., Zimmermann-Kogadeeva, M., Wegmann, R. & Goodman, A. L. Mapping human microbiome drug metabolism by gut bacteria and their genes. Nature 570, 462-467 (2019).
8. Koppel, N., Maini Rekdal, V. & Balskus, E. P. Chemical transformation of xenobiotics by the human gut microbiota. Science 356, (2017).
9. Balaich, J. et al. The human microbiome encodes resistance to the antidiabetic drug acarbose. Nature 600, 110-115 (2021).
10. Haiser, H. J. et al. Predicting and manipulating cardiac drug inactivation by the human gut bacterium Eggerthella lenta. Science 341, 295-298 (2013).
11. Rekdal, V. M., Bess, E. N., Bisanz, J. E., Turnbaugh, P. J. & Balskus, E. P. Discovery and inhibition of an interspecies gut bacterial pathway for Levodopa metabolism. Science 364, eaau6323 (2019).
12. Haiser, H. J., Seim, K. L., Balskus, E. P. & Turnbaugh, P. J. Mechanistic insight into digoxin inactivation by Eggerthella lenta augments our understanding of its pharmacokinetics. Gut Microbes 5, 233-238 (2014).
13. Lee, J. W. J. et al. Multi-omics reveal microbial determinants impacting responses to biologic therapies in inflammatory bowel disease. Cell Host Microbe 29, 1294-1304.e4 (2021).
14. Forslund, S. K. et al. Combinatorial, additive and dose-dependent drug-microbiome associations. Nature 600, 500-505 (2021).
15. van Hogezand, R. A. et al. Bacterial acetylation of 5-aminosalicylic acid in faecal suspensions cultured under aerobic and anaerobic conditions. Eur J Clin Pharmacol 43, 189-192 (1992).
16. Dull, B. J., Salata, K. & Goldman, P. Role of the intestinal flora in the acetylation of sulfasalazine metabolites. Biochemical Pharmacology 36, 3772-3774 (1987).
17. van Hogezand, R. A. et al. Double-blind comparison of 5-aminosalicylic acid and acetyl-5-aminosalicylic acid suppositories in patients with idiopathic proctitis. Aliment Pharmacol Ther 2, 33-40 (1988).
18. Sandborn, W. J. & Hanauer, S. B. Systematic review: the pharmacokinetic profiles of oral mesalazine formulations and mesalazine pro-drugs used in the management of ulcerative colitis. Aliment Pharmacol Ther 17, 29-42 (2003).
19. Ireland, A., Priddle, J. D. & Jewell, D. P. Comparison of 5-aminosalicylic acid and N-acetylaminosalicylic acid uptake by the isolated human colonic epithelial cell. Gut 33, 1343-1347 (1992).
20. Deloménie, C. et al. Identification and functional characterization of arylamine N-acetyltransferases in eubacteria: evidence for highly selective acetylation of 5-aminosalicylic acid. J. Bacteriol. 183, 3417-3427 (2001).
21. Westwood, I. M. et al. Expression, purification, characterization and structure of Pseudomonas aeruginosa arylamine N-acetyltransferase. Biochem J 385, 605-612 (2005).
22. Lloyd-Price, J. et al. Multi-omics of the gut microbial ecosystem in inflammatory bowel diseases. Nature 569, 655 (2019).
23. Beghini, F. et al. Integrating taxonomic, functional, and strain-level profiling of diverse microbial communities with bioBakery 3. eLife 10, e65088 (2021).
24. Hafferty, J. D. et al. Self-reported medication use validated through record linkage to national prescribing data. J Clin Epidemiol 94, 132-142 (2018).
25. Akobeng, A. K., Zhang, D., Gordon, M. & MacDonald, J. K. Oral 5-aminosalicylic acid for maintenance of medically-induced remission in Crohn's disease. Cochrane Database of Systematic Reviews (2016) doi:10.1 002/14651 858.CD003715.pub3.
26. Neshich, I. A., Kiyota, E. & Arruda, P. Genome-wide analysis of lysine catabolism in bacteria reveals new connections with osmotic stress resistance. The ISME Journal 7, 2400-2410 (2013).
27. Sell, D. R., Strauch, C. M., Shen, W. & Monnier, V. M. 2-aminoadipic acid is a marker of protein carbonyl oxidation in the aging human skin: effects of diabetes, renal failure and sepsis. Biochem J 404, 269-277 (2007).
28. Harary, I. Bacterial degradation of nicotinic acid. Nature 177, 328-329 (1956).
29. Li, J. et al. Niacin ameliorates ulcerative colitis via prostaglandin D2-mediated D prostanoid receptor 1 activation. EMBO Mol Med 9, 571-588 (2017).
30. Franzosa, E. A. et al. Gut microbiome structure and metabolic activity in inflammatory bowel disease. Nature Microbiology 4, 293 (2019).
31. Zhang, Y., Thompson, K. N., Huttenhower, C. & Franzosa, E. A. Statistical approaches for differential expression analysis in metatranscriptomics. Bioinformatics 37, i34-i41 (2021).
32. Devos, D. & Valencia, A. Practical limits of function prediction. Proteins: Structure, Function, and Bioinformatics 41, 98-107 (2000).
33. Fitzgerald, C. B. et al. Comparative analysis of Faecalibacterium prausnitzii genomes shows a high level of genome plasticity and warrants separation into new species-level taxa. BMC Genomics 19, 931 (2018).
34. Rousseaux, C. et al. Intestinal antiinflammatory effect of 5-aminosalicylic acid is dependent on peroxisome proliferator-activated receptor-γ. J Exp Med 201, 1205-1215 (2005).
35. Modis, Y. & Wierenga, R. K. Crystallographic analysis of the reaction pathway of Zoogloea ramigera biosynthetic thiolase. J Mol Biol 297, 1171-1182 (2000).
36. Kim, S. et al. Redox-switch regulatory mechanism of thiolase from Clostridium acetobutylicum. Nat Commun 6, 8410 (2015).
37. Mathieu, M. et al. The 1.8 Å crystal structure of the dimeric peroxisomal 3-ketoacyl-CoA thiolase of Saccharomyces cerevisiae: implications for substrate binding and reaction mechanism 11 Edited by R. Huber. Journal of Molecular Biology 273, 714-728 (1997).
38. Hyams, J. S. et al. Clinical and biological predictors of response to standardised paediatric colitis therapy (PROTECT): a multicentre inception cohort study. Lancet 393, 1708-1720 (2019).
39. Ananthakrishnan, A. N. Environmental Triggers for Inflammatory Bowel Disease. Curr Gastroenterol Rep 15, 302 (2013).
40. Ricart, E. et al. N-acetyltransferase 1 and 2 genotypes do not predict response or toxicity to treatment with mesalamine and sulfasalazine in patients with ulcerative colitis. Am. J. Gastroenterol. 97, 1763-1768 (2002).
41. Yee, J. et al. The association between NAT2 acetylator status and adverse drug reactions of sulfasalazine: a systematic review and meta-analysis. Sci Rep 10, 3658 (2020).
42. Lück, H., Kinzig, M., Jetter, A., Fuhr, U. & Sörgel, F. Mesalazine pharmacokinetics and NAT2 phenotype. Eur J Clin Pharmacol 65, 47-54 (2009).
43. Ha, C. Y., Newberry, R. D., Stone, C. D. & Ciorba, M. A. Patients with Late Adult Onset Ulcerative Colitis Have Better Outcomes than Those with Early Onset Disease. Clin Gastroenterol Hepatol 8, 682-687.e1 (2010).
44. Huberts, D. H. E. W. & van der Klei, I. J. Moonlighting proteins: an intriguing mode of multitasking. Biochim Biophys Acta 1803, 520-525 (2010).
45. Hong, J., Park, W., Seo, H., Kim, I.-K. & Kim, K.-J. Crystal structure of an acetyl-CoA acetyltransferase from PHB producing bacterium Bacillus cereus ATCC 14579. Biochemical and Biophysical Research Communications 533, 442-448 (2020).
46. Maier, L. et al. Extensive impact of non-antibiotic drugs on human gut bacteria. Nature 555, 623-628 (2018).
47. Wallace, B. D. et al. Alleviating Cancer Drug Toxicity by Inhibiting a Bacterial Enzyme. Science 330, 831-835 (2010).
48. De Vos, M. et al. Concentrations of 5-ASA and Ac-5-ASA in human ileocolonic biopsy homogenates after oral 5-ASA preparations. Gut 33, 1338-1342 (1992).
49. Lohman, B. K., Weber, J. N. & Bolnick, D. I. Evaluation of TagSeq, a reliable low-cost alternative for RNAseq. Molecular Ecology Resources 16, 1315-1321 (2016).
50. Suzek, B. E. et al. UniRef clusters: a comprehensive and scalable alternative for improving sequence similarity searches. Bioinformatics 31, 926-932 (2015).
51. Oksanen, J. et al. The vegan package. Community ecology package 10, 719 (2007).
52. Wickham, H. ggplot2: elegant graphics for data analysis. (springer, 2016).
53. Wang, M. et al. Mass spectrometry searches using MASST. Nature biotechnology 38, 23-26 (2020).
54. Aron, A. T. et al. Reproducible molecular networking of untargeted mass spectrometry data using GNPS. Nat Protoc 15, 1954-1991 (2020).
55. Mehta, R. S. et al. Dietary Patterns and Risk of Colorectal Cancer: Analysis by Tumor Location and Molecular Subtypes. Gastroenterology 152, 1944-1953.e1 (2017).
56. Bar, N. et al. A reference map of potential determinants for the human serum metabolome. Nature 588, 135-140 (2020).
57. Groemping, U. & Matthias, L. Package ‘relaimpo’. Relative Importance of Regressors in Linear Models; R Fundation for Statstical Computing: Vienna, Austria (2021).
58. Bustion, A., Agrawal, A., Turnbaugh, P. J. & Pollard, K. S. A novel in silico method employs chemical and protein similarity algorithms to accurately identify chemical transformations in the human gut microbiome. 2022.08.02.502504 Preprint at doi.org/10.1101/2022.08.02.502504 (2022).
59. Franzosa, E. A. et al. Relating the metatranscriptome and metagenome of the human gut. Proc. Natl. Acad. Sci. U.S.A. 111, E2329-2338 (2014).
60. Pinheiro, J. et al. Package ‘nlme’. Linear and nonlinear mixed effects models, version 3, (2017).
61. Kenny, D. J. et al. Cholesterol Metabolism by Uncultured Human Gut Bacteria Influences Host Cholesterol Level. Cell Host Microbe 28, 245-257.e6 (2020).
62. The UniProt Consortium. UniProt: the universal protein knowledgebase in 2021. Nucleic Acids Research 49, D480-D489 (2021).
63. Thompson, J. D., Higgins, D. G. & Gibson, T. J. CLUSTAL W: improving the sensitivity of progressive multiple sequence alignment through sequence weighting, position-specific gap penalties and weight matrix choice. Nucleic Acids Research 22, 4673-4680 (1994).
64. Henikoff, S. & Henikoff, J. G. Amino acid substitution matrices from protein blocks. Proceedings of the National Academy of Sciences 89, 10915-10919 (1992).
65. Hunter, S. et al. InterPro: the integrative protein signature database. Nucleic Acids Research 37, D211-D215 (2009).
66. Kitts, P. A. et al. Assembly: a resource for assembled genomes at NCBI. Nucleic Acids Res 44, D73-80(2016).
67. Buchfink, B., Xie, C. & Huson, D. H. Fast and sensitive protein alignment using DIAMOND. Nat Methods 12, 59-60 (2015).
68. Dawwas, G. K. et al. Prevalence and Factors Associated With Fecal Urgency Among Patients With Ulcerative Colitis and Crohn's Disease in the Study of a Prospective Adult Research Cohort With Inflammatory Bowel Disease. Crohn's & Colitis 360 3, otab046 (2021).
69. Raffals, L. E. et al. The Development and Initial Findings of A Study of a Prospective Adult Research Cohort with Inflammatory Bowel Disease (SPARC IBD). Inflammatory Bowel Diseases (2021) doi:10.1093/ibd/izab071.
70. Carey, V. J., and 4.4), T. S. L. (R port of versions 3 13, src/d*), C. M. (LINPACK routines in & updates), B. R. (R port of version 4 13 and. gee: Generalized Estimation Equation Solver. (2022).
71. Viechtbauer, W. Conducting Meta-Analyses in R with the meta for Package. Journal of Statistical Software 36, 1-48 (2010).
72. Garcia-Closas, M. et al. NAT2 slow acetylation and GSTM1 null genotypes increase bladder cancer risk: results from the Spanish Bladder Cancer Study and meta-analyses. Lancet 366, 649-659 (2005).
73. Chan, S. L. et al. Association and clinical utility of NAT2 in the prediction of isoniazid-induced liver injury in Singaporean patients. PLoS One 12, e0186200 (2017).
74. Selinski, S. et al. Genotyping NAT2 with only two SNPs (rs1041983 and rs1801280) outperforms the tagging SNP rs1495741 and is equivalent to the conventional 7-SNP NAT2 genotype. Pharmacogenetics and Genomics 21, 673-678 (2011).
75. XDS Package. xds.mr.mpg.de/.
76. Liebschner, D. et al. Macromolecular structure determination using X-rays, neutrons and electrons: recent developments in Phenix. Acta Crystallogr D Struct Bio/75, 861-877 (2019).
77. Coot. www2.mrc-Imb.cam.ac.uk/personal/pemsley/coot/.
78. Pettersen, E. F. et al. UCSF Chimera—a visualization system for exploratory research and analysis. J Comput Chem 25, 1605-1612 (2004).
79. Ding, R. W. et al. Pharmacokinetics of nicotinic acid—salicylic acid interaction. Clinical Pharmacology & Therapeutics 46, 642-647 (1989).
80. Santoru, M. L. et al. Cross sectional evaluation of the gut-microbiome metabolome axis in an Italian cohort of IBD patients. Sci Rep 7, 9523 (2017).

While the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

We claim:

1. A method of determining whether a patient diagnosed with inflammatory bowel disease (IBD) will respond to 5-ASA therapy, the method comprising:
(a) obtaining a stool sample from the patient,
(b) analyzing the sample for the presence of microbial acetyltransferase genes capable of converting 5-ASA to the clinically ineffective N-acetyl 5-ASA,
(c) if microbial acetyltransferase genes are not present in the sample, the patient will respond to 5-ASA therapy and such therapy should be administered; if microbial acetyltransferase genes are present in the sample, the patient will not respond to 5-ASA therapy and a second line therapy should be administered.

2. The method according to claim 1, wherein the microbial acetyltransferase genes are three or more of the following genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii.

3. The method of claim 1, wherein IBD is ulcerative colitis.

4. The method of claim 1, wherein IBD is Crohn's Disease.

5. The method of claim 1, wherein the second line therapy is selected from a biologic for treating IBD.

6. The method of claim 1, wherein the second line therapy is selected from an immunomodulator for treating IBD.

7. The method of claim 1, wherein analyzing comprises qPCR.

8. A method for treating a patient having IBD comprising administering to the patient a biologic in an amount effective to treat IBD when a stool sample from the patient comprises microbial acetyltransferase genes capable of converting 5-ASA to N-acetyl 5-ASA.

9. The method of claim 8, wherein IBD is ulcerative colitis.

10. The method of claim 8, wherein IBD is Crohn's Disease.

11. The method of claim 8, wherein the biologic for treating IBD is adalimumab, vedolizumab, or infliximab.

12. A method for treating a patient having IBD comprising administering to the patient an immunomodulator in an amount effective to treat IBD when a stool sample from the patient comprises microbial acetyltransferase genes capable of converting 5-ASA to N-acetyl 5-ASA.

13. The method of claim 12, wherein IBD is ulcerative colitis.

14. The method of claim 12, wherein IBD is Crohn's Disease.

15. The method of claim 12, wherein the immunomodulator for treating IBD is 6-mercaptopurine or tofacitinib.

16. A method for treating a patient having IBD comprising administering to the patient 5-aminosalicylic acid (5-ASA) in an amount effective to treat IBD when microbial acetyltransferase genes are not present in a stool sample from the patient.

17. The method of claim 16, wherein IBD is ulcerative colitis.

18. The method of claim 16, wherein IBD is Crohn's Disease.

19. The method according to claim 16, wherein the microbial acetyltransferase genes are three or more of the following genes:
R6CZ24—“Acetyl-CoA acetyltransferase” from Firmicutes bacterium CAG:176
T5S060—“Acetyl-CoA C-acetyltransferase” from Ruminococcaceae bacterium D16
R5CY66—“Acetyl-CoA acetyltransferase” from Firmicutes bacterium CAG:83
C7H1G6—“Acetyltransferase, GNAT family” from Faecalibacterium prausnitzii.

20. The method of claim 16, wherein analyzing for the presence or absence of microbial acetyltransferase genes comprises qPCR.
